SEQUENCE LISTING (24 sequences)

SEQ ID NO: 1 - DNA, 1010 bp, Homo sapiens
  source 1..1010: mol_type = genomic DNA
tgagcggataacaattcccctctagaaataattttgtttaactttaagaaggagatataccatgtacagctgcaacgtgtgtgggaaagcttttgtactttcgcgccacttgaaccgccacttacgtgtacatcgtcgcgcgactggtagcgggatggtagctggcatgctgggtcttcgcgaagagaaaccagccgttccggaagcatcggcgtcgcccaagcaacgtcgcagcatcatccgcgaccgcggccccatgtacgatgatccgactttacctgaaggttggacacgtaaactgaagcaacgtaagagcgggcgttcagcggggaagtatgatgtttatttgattaacccacaggggaaggctttccgctctaaggttgagcttatcgcctacttcgagaaagtgggagatacctcgctggatccgaacgatttcgattttacagtcacaggccgtggctccccatcccgccgcgagcagaagcctccgaaaaaaccaaaatctcctaaggcgccaggcactggacgtggtcgcgggcgtcctaagggcagcggtaccggacgcccaaaagcagctgcctccgaaggagtacaagtgaaacgtgttttagaaaagagtccaggaaaacttgttgtgaaaatgccctttcaggcgagcccggggggcaaaggcgagggcggcggcgccacaacatctgctcaggtaatggttatcaaacgtcctggccgtaagcgtaaagccgaagcggaccctcaagcaattccaaagaaacgcggtcgtaaaccaggctcggtagtagccgctgcggcggctgaagctaaaaagaaggccgttaaggagtctagcattcgcagtgtgcacgagacagttttaccgatcaagaaacgtaagactcgtgagaccgttggctctagcgggtcctcaggtcttccggaaactggagggtggagtcacccacaattcgagaaataacggatccgaattcgagcgccgtcgacaagct

SEQ ID NO: 2 - DNA, 179 bp, Homo sapiens
  source 1..179: mol_type = genomic DNA
tgagcggataacaattcccctctagaaataattttgtttaactttaagaaggagatataccatggtagctggcatgctgggtcttcgcgaagagaaaccagccgttccggaagcatcggcgtcgcccaagcaacgtcgcagcatcatccgcgaccgcggccccatgtacgatgatccga

SEQ ID NO: 3 - DNA, 224 bp, Homo sapiens
  source 1..224: mol_type = genomic DNA
tgagcggataacaattcccctctagaaataattttgtttaactttaagaaggagatataccatgtacggtcgcaaaaaacgccgccaacgtcgtcgcggttccggtatggtagctggcatgctgggtcttcgcgaagagaaaccagccgttccggaagcatcggcgtcgcccaagcaacgtcgcagcatcatccgcgaccgcggccccatgtacgatgatccga

SEQ ID NO: 4 - DNA, 28 bp, Homo sapiens
  source 1..28: mol_type = genomic DNA
tttcgattttatggtcacaggccgtggc

SEQ ID NO: 5 - DNA, 18 bp, Homo sapiens
  source 1..18: mol_type = genomic DNA
tcgttcggatccagcgag

SEQ ID NO: 6 - DNA, 28 bp, Homo sapiens
  source 1..28: mol_type = genomic DNA
gacagttttactgatcaagaaacgtaag

SEQ ID NO: 7 - DNA, 18 bp, Homo sapiens
  source 1..18: mol_type = genomic DNA
tcgtgcacactgcgaatg

SEQ ID NO: 8 - protein, 274 aa, Homo sapiens
MVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVGSSGSSGLPETGGWSHPQFEK

SEQ ID NO: 9 - protein, 305 aa, Homo sapiens
MYSCNVCGKAFVLSRHLNRHLRVHRRATGSGMVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVGSSGSSGLPETGGWSHPQFEK

SEQ ID NO: 10 - protein, 289 aa, Homo sapiens
MYGRKKRRQRRRGSGMVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVGSSGSSGLPETGGWSHPQFEK

SEQ ID NO: 11 - protein, 305 aa, Homo sapiens
MYSCNVCGKAFVLSRHLNRHLRVHRRATGSGMVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFMVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVGSSGSSGLPETGGWSHPQFEK

SEQ ID NO: 12 - protein, 305 aa, Homo sapiens
MYSCNVCGKAFVLSRHLNRHLRVHRRATGSGMVAGMLGLREEKPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLLIKKRKTRETVGSSGSSGLPETGGWSHPQFEK

SEQ ID NO: 13 - DNA, 22 bp, Homo sapiens
  source 1..22: mol_type = other DNA
  modified_base 1: mod_base = OTHER; note = 5(6)-Carboxyfluorescein
  modified_base 9: mod_base = OTHER; note = methyl deoxycytosine
ccctataacggaattcataatg

SEQ ID NO: 14 - DNA, 22 bp, Homo sapiens
  source 1..22: mol_type = other DNA
  modified_base 13: mod_base = OTHER; note = methyl deoxycytosine
cattatgaattccgttataggg

SEQ ID NO: 15 - DNA, 22 bp, Homo sapiens
  source 1..22: mol_type = other DNA
  modified_base 1: mod_base = OTHER; note = 5(6)-Carboxyfluorescein
ccctataacggaattcataatg

SEQ ID NO: 16 - DNA, 22 bp, Homo sapiens
  source 1..22: mol_type = other DNA
cattatgaattccgttataggg

SEQ ID NO: 17 - DNA, 22 bp, Homo sapiens
  source 1..22: mol_type = other DNA
ctgaccactcagtacatcatcc

SEQ ID NO: 18 - DNA, 21 bp, Homo sapiens
  source 1..21: mol_type = other DNA
ctcctgtctccctatcctctt

SEQ ID NO: 19 - DNA, 22 bp, Homo sapiens
  source 1..22: mol_type = other DNA
agggtctggctattccttatct

SEQ ID NO: 20 - DNA, 23 bp, Homo sapiens
  source 1..23: mol_type = other DNA
ccttgcaccttctcacagtatag

SEQ ID NO: 21 - DNA, 59 bp, synthetic construct
  source 1..59: mol_type = other DNA
agcgaggaaggtcagaagcttggcaatccggtactggccaccatggtgagcaagggcga

SEQ ID NO: 22 - DNA, 43 bp, synthetic construct
  source 1..43: mol_type = other DNA
ggcggaaagccgtgagaattcgccttgtacagctcgtccatgc

SEQ ID NO: 23 - DNA, 54 bp, synthetic construct
  source 1..54: mol_type = other DNA
ccggtacctgagctcgctagctttctgtacagaattttaagtatctgacaattt

SEQ ID NO: 24 - DNA, 42 bp, synthetic construct
  source 1..42: mol_type = other DNA
cagtaccggattgccaagcttctgaccttcctcgctccattg

(19) United States
(12) Patent Application Publication
(10) Pub. No.: US 2025/0092101 A1
(43) Pub. Date: Mar. 20, 2025

(54) Nuclear Delivery and Transcriptional Repression with a Cell-penetrant MeCP2

(71) Applicant: The Regents of the University of California, Oakland, CA (US)

(72) Inventors: Alanna Schepartz Shrader, Berkeley, CA (US); Xizi Zhang, Berkeley, CA (US)

(73) Assignee: The Regents of the University of California, Oakland, CA (US)

(21) Appl. No.: 18/964,332

(22) Filed: Nov. 29, 2024

Related U.S. Application Data
(63) Continuation of application No. PCT/US23/66099, filed on Apr. 23, 2023.
(60) Provisional application No. 63/348,683, filed on Jun. 3, 2022.

(51) Int. Cl. C07K 14/47; A61K 38/00

(52) U.S. Cl. CPC C07K 14/4702; A61K 38/00; C07K 2319/81

(57) ABSTRACT

Methods and compositions for nuclear delivery and transcriptional repression employ a cell-penetrant MeCP2 fusion protein.

### CROSS-REFERENCES TO RELATED APPLICATIONS

This application is a continuation of PCT/US23/66099, filed Apr. 23, 2023, which claims priority to U.S. Provisional Application No. 63/348,683, filed Jun. 3, 2022, the disclosures of which are hereby incorporated by reference in its entirety for all purposes.

### REFERENCE TO A SEQUENCE LISTING

A Sequence Listing in XML format is incorporated by reference into the specification. The name of the XML file containing the Sequence Listing is B22-131-2US.xml. The XML file is 27,023 bytes and was created on Nov. 28, 2024.

### INTRODUCTION

Methyl-CpG-binding-protein 2 (MeCP2) is an abundant nuclear protein expressed in all cell types, especially neurons1. Mutations in the MECP2 gene cause Rett syndrome (RTT), a severe and incurable neurological disorder that disproportionately affects young girls2. Many potential RTT treatments are under development12, but no disease modifying treatment yet exists. Two features of RTT etiology render therapeutic development especially challenging. The first is that more than 850 different mutations in the MECP2 gene13 account for >95% of classical RTT cases14; this feature complicates approaches based on gene-editing15-17. The second is that excess MeCP2 protein causes MeCP2 duplication syndrome18 which itself causes progressive neurological disorders; this feature complicates approaches that rely on gene delivery19,6,20-22. Since 2007, several studies have demonstrated that restoring MeCP2 expression can phenotypically reverse RTT-like symptoms in male and female MeCP2-deficient mice3-5. These rescue experiments provide evidence that dose-dependent, nuclear delivery of functional MeCP2 protein could provide a novel treatment modality. Although the concentration of MeCP2 varies between cell types, its primary function is to engage the NCoR/SMRT co-repressor complex in a methylated DNA-dependent manner23. Thus, in order to be effective, MeCP2 protein must reach the nucleus intact, transcriptionally active, and in the high nanomolar to low micromolar concentration range 1,24.

Previous efforts to deliver MeCP2 protein suffered from low delivery efficiency and significant cargo degradation25-29. We showed previously that the mini-protein ZF5.37-11 is taken up by the endosomal pathway and released efficiently into the cytosol and nuclei of live cells8, alone and when fused to certain protein cargos. Proteins successfully delivered using ZF5.3 include the model protein SNAP-tag9, the metabolic enzyme argininosuccinate synthetase11, and the proximity labeling tool APEX29. These proteins differ in molecular weight, stoichiometry, isoelectric point, and the presence of bound cofactors. In all cases evaluated, the protein that reached the cytosol was fully intact as judged by Western blot analysis of isolated cytosolic fractions free of detectable endosomal contamination, and the delivery efficiencies were 2-10-fold9 higher than seen with canonical or cyclic peptides30. Mechanistic studies confirm that ZF5.3 relies on the endocytic pathway to reach the cell interior7, and that endosomal escape into the cytosol demands a functional homotypic fusion and protein sorting (HOPS) complex10.

U.S. Ser. No. 10/227,384 discloses ZF5.3 fusion proteins; U.S. Pat. No. 8,226,930 discloses synthetic MeCP2 sequences for protein substitution therapy

### SUMMARY OF THE INVENTION

ZF-tMeCP2 conjugates bind DNA in a methylation-dependent manner and reach the nucleus of model cell lines intact at concentrations above 700 nM. When delivered to live cells, ZF-tMeCP2 engages the NCoR/SMRT co-repressor complex and selectively represses transcription from methylated promoters.

The invention provides methods and compositions for nuclear delivery and transcriptional repression with a cell-penetrant MeCP2.

In an aspect the invention provides a cell-penetrant ZF5.3-MeCP2 fusion protein comprising a ZF5.3 moiety and a methyl-CpG binding protein 2 (MeCP2) moiety, the MeCP2 moiety comprising a methyl-CpG binding domain (MBD) and a NCoR/SMRT interaction domain (NID).

In embodiments:

the ZF5.3 moiety is N-terminal with respect to the MeCP2 moiety;

the MBD and NID comprise residues 72-173 and 272-312 of MeCP2, respectively, with respect to the mouse e2 isoform;

the ZF5.3 moiety is N-terminal with respect to the MeCP2 moiety, and the MBD and NID comprise residues 72-173 and 272-312 of MeCP2, respectively, with respect to the mouse e2 isoform;

the fusion protein reaches the nucleus of the cell intact and transcriptionally active, as measured in human osteosarcoma (clone Saos-2) or hamster chinese ovary (clone CHO-K1) cells; and/or

the fusion protein reaches the nucleus of the cell at at least 100 or 500 nM.

In an aspect the invention provides a method of introducing MeCP2 activity in a host in need thereof, comprising delivering the MeCP2 activity in the form of a cell-penetrant ZF5.3-MeCP2 fusion protein herein.

In an aspect the invention provides a method of treating Rett syndrome (RTT), comprising administering to a person in need thereof a cell-penetrant ZF5.3-MeCP2 fusion protein herein.

The invention encompasses all combinations of the particular embodiments recited herein, as if each combination had been laboriously recited.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Example: Dose-Dependent Nuclear Delivery and Transcriptional Repression with a Cell-Penetrant MeCP2

Here we use chemical biology, cell biology, biophysics, and biochemistry tools to qualitatively and quantitatively assess the nuclear delivery and function of MeCP2 conjugates of ZF5.3 and Tat. Although both conjugates bind DNA in a methylation-dependent manner in vitro and appear to reach the nucleus as judged by fluorescence-based methods, biochemical fractionation studies reveal that only the conjugate with ZF5.3 remains fully intact within the nucleus. When delivered to live cells, the conjugate between ZF5.3 and MeCP2 effectively engages the NCoR/SMRT co-repressor complex and selectively represses transcription from methylated promoters. Efficient nuclear delivery relies on HOPS-dependent endosomal fusion. By contrast, the Tat conjugate of MeCP2 is degraded within the nucleus, is not selective for methylated promoters, and trafficks in a HOPS-independent manner. The results described demonstrate the feasibility of a HOPS-dependent portal for delivering functional macromolecules to the cell interior using the cell-penetrant mini-protein ZF5.3.

### Design, Purification, and Characterization of MeCP2 Variants

Full-length murine MeCP2 (MeCP2-e2) contains 484 amino acids (52 kD)⁶. MeCP2(ΔNC) (referred to henceforth as tMeCP2) is shorter (27 kDa, 253 aa) but mirrors MeCP2 in its interactions with methylated DNA and the NCoR/SMRT complex, and Mecp2-null male mice display a near-normal phenotype upon expression of MeCP2(ΔNC)6. We generated fusion proteins containing a single copy of ZF5.37 or Tat₄₇₋₅₇ 31 followed by the complete sequence of tMeCP2. Each fusion protein also contained a sortase recognition motif (6 aa) to enable site-specific fluorophore conjugation and a Strep-tag II sequence (8 aa) to enable affinity purification. We also prepared two tMeCP2 variants with substitutions that alter function. The first is T158M tMeCP2, with a methyl-CpG-binding domain (MBD) mutation that reduces specific DNA binding32,33 and is seen commonly in RTT patients14. The second is P302L tMeCP2, which has a diminished ability to engage the TBLR1 subunit of the NCoR/SMRT repressor complex34. All tMeCP2 variants were expressed in E. coli, purified to >95% homogeneity, and characterized using Western blots and LC/MS. Variants carrying a fluorescent label were generated using sortase-A and a GGGK-lissamine rhodamine B (Rho) co-reagent as previously described9. Circular dichroism (CD) analysis of all tMeCP2 variants confirmed that the conjugation of ZF5.3 and Tat₄₇₋₅₇ had minimal effect on protein secondary structure. Consistent with previous reports for full-length MeCP235, all tMeCP2 variants show high levels of intrinsic disorder (60%) in the absence of DNA.

Purified tMeCP2 Proteins are Active In Vitro

MeCP2 functions like a bridge to repress transcription from methylated promoters23. The N-terminal methyl-CpG-binding domain (MBD) interacts with methylated DNA32 while the C-terminal transcriptional-repressor domain (TRD) domain engages NCoR1/2 co-repressor partners34,36. To establish whether the tMeCP2 proteins studied here retain these functions in vitro, we measured their affinities for methylated and non-methylated DNA oligonucleotides using a fluorescence polarization assay and used immunoprecipitation methods to assess interactions with co-receptor proteins in lysates. Fluorescence polarization analysis was performed with a 22 bp double-stranded, fluorescein-tagged, DNA oligonucleotide containing a methylated or non-methylated cytosine. tMeCP2 interacts with methylated DNA with a K_{D} of 15 nM and a 15-fold preference for methylated versus non-methylated DNA. ZF-tMeCP2 interacts with methylated DNA with a K_{D} of 55 nM and a 6-fold preference for methylated DNA. The conjugate of Tat₄₇₋₅₇ and tMeCP2 (Tat-tMeCP2) interacts with both methylated DNA (K_{D}=5 nM) and non-methylated DNA (K_{D}=25 nM) more favorably than tMeCP2 and ZF-tMeCP2 with a 5-fold preference for methylated DNA. As expected, ZF-tMeCP2(T158M) binds poorly to both methylated (K_{D}=210 nM) and non-methylated (K_{D}=1.1 μM) DNA when compared to tMeCP2. Although the K_{D} describing the interaction of tMeCP2 with methylated DNA has not previously been determined, reported values for full-length MeCP2 fall in the range of 36-130 nM with a 2-33 fold preference for methylated DNA37,38.

To further probe the function of purified ZF-tMeCP2 in vitro, we used affinity pull-down assays to evaluate its interactions with the NCoR/SMRT co-repressor complex in nuclear lysates of NIH3T3 cells6. Lysates36 were incubated overnight at 4° C. with 1.5 μM of ZF-tMeCP2, Tat-tMeCP2, or tMeCP2; ZF-tMeCP2(P302L) was used as a negative control. Each tMeCP2 variant was extracted from the lysates using streptavidin-coated beads, and the identities and relative levels of bound NCoR/SMRT subunits (NCoR1, HDAC3, and TBL1/TBLR1) were evaluated using Western blots. These blots revealed that tMeCP2, ZF-tMeCP2, and Tat-tMeCP2 remain intact after an overnight incubation with lysates at 4° C. and effectively sequester HDAC3, TBL1/TBLR1, NCoR1 from NIH3T3 nuclear cell lysates. In all cases, there was little or no evidence of interaction with ZF-tMeCP2(P302L). Taken together, these two in vitro assays confirm that purified ZF-tMeCP2 retains the core functions of MeCP2: selective recognition of methylated DNA and the ability to engage the NCoR/SMRT co-repressor complex.

### Efficient Delivery of ZF-tMeCP2 to the Nuclei of Saos-2 and CHO-K1 Cells

Next, we made use of three fluorescence-based methods and two model cell lines to evaluate the overall uptake of each tMeCP2 variant and specifically how much protein trafficked to the nucleus, the site of MeCP2 function. Human osteosarcoma (Saos-2) cells were incubated for 1 hr with rhodamine-tagged tMeCP2-Rho, ZF-tMeCP2-Rho, Tat-tMeCP2-Rho, or ZF-tMeCP2(T158M)-Rho at concentrations between 0.5 μM and 2 μM. When visualized using 2D confocal microscopy, cells treated individually with each of the four tMeCP2-Rho variants showed bright punctate intracellular fluorescence, while no fluorescence was observed in non-treated cells. Saos-2 cells treated with ZF-tMeCP2-Rho, Tat-tMeCP2-Rho, and ZF-tMeCP2(T158M)-Rho also showed evidence of intra-nuclear fluorescence at concentrations as low as 1 μM, while cells treated tMeCP2-Rho did not, even at 2 μM concentration. When visualized as 3D z-stacks, cells treated with ZF-tMeCP2-Rho, Tat-tMeCP2-Rho, and ZF-tMeCP2(T158M)-Rho differed in intra-nuclear localization. Cells treated with ZF-tMeCP2-Rho and Tat-tMeCP2-Rho showed an even distribution of rhodamine fluorescence in Hoechst-positive, DNA-rich regions, whereas cells treated with ZF-tMeCP2(T158M)-Rho showed aggregated rhodamine signal in small discrete regions resembling nucleoli. This observation aligns with previous reports that truncation of the entire MBD or T158M mutation resulted in MeCP2 relocalization to the nucleolus40,41.

We next used fluorescence correlation spectroscopy (FCS) to quantitatively track the cytosolic and nuclear distribution of each tMeCP2-Rho conjugate in Saos-2 and CHO-K1 cells. FCS is a single-molecule technique that deconvolutes the time-dependent change in fluorescence in a small cytosolic or nuclear volume to establish both average intracellular concentration as well as diffusion time42,43. FCS analysis of Saos-2 cells revealed that all tMeCP2-Rho conjugates localize more significantly to the nucleus than the cytosol, as established qualitatively by confocal microscopy. Localization to the nucleus is dose-dependent between 500 nM and 1 μM, even for tMeCP2-Rho. At low treatment concentrations (0.5 μM), the FCS-determined mean nuclear delivery efficiencies of tMeCP2-Rho and Tat-tMeCP2-Rho were lower than either ZF-tMeCP2-Rho (2.3-fold) or ZF-tMeCP2(T158M)-Rho (1.6-fold). At 1 μM, ZF-tMeCP2-Rho, ZF-tMeCP2(T158M)-Rho, and Tat-tMeCP2-Rho reach the nucleus more efficiently (2.0-2.3-fold) than tMeCP2-Rho, with localization efficiency increasing in the order: tMeCP2-Rho<Tat-tMeCP2-Rho<ZF-tMeCP2-Rho˜ZF-tMeCP2(T158M)Rho.

We note that while the conjugation of ZF5.3 to tMeCP2 resulted in a smaller fold-improvement in nuclear or cytosolic delivery than previously reported examples (improvements between 39,11-329-fold), the mean nuclear concentration of ZF-tMeCP2 established in Saos-2 cells after a 1 h incubation with 1 μM protein (709±69 nM) is the highest intracellular concentration yet measured for a delivered protein9,11. The high concentration of ZF-tMeCP2 that reaches the nucleus may result from the higher intrinsic permeability of tMeCP2 itself in comparison to other proteins when evaluated under comparable conditions (argininosuccinate synthetase: 77±30 nM, SNAP-tag: 2±1 nM)^{9,11}. While further studies are needed to establish the factors that lead to high intrinsic permeability, we note that tMeCP2 is characterized by both a high pI (10.78) and high levels (60%) of intrinsic disorder as judged by CD.

### Flow Cytometry as a High-Throughput Alternative to FCS for Evaluating Nuclear Delivery En Masse

The nuclear fluorescence of cells treated with 2 μM ZF-tMeCP2-Rho, Tat-tMeCP2-Rho and ZF-tMeCP2(T158M)-Rho was too high to be reliably measured by FCS42,43. Although the total fluorescence of intact cells measured using flow cytometry does not reliably quantify how much material escapes from the endosomal pathway9, procedures to isolate and sort nuclei on the basis of fluorescence are well known45. We wondered whether the fluorescence of nuclei isolated from cells treated with tMeCP2-Rho variants would correlate with the nuclear concentration measured in intact cells using FCS. If such a correlation was observed, then flow cytometry would provide an extremely high-throughput and rapid alternative to FCS for quantifying delivery of fluorescently tagged material to the nucleus.

Thus, we isolated intact nuclei from Saos-2 cells after treatment with 0.5-1.0 μM tMeCP2-Rho variants and 2 μM tMeCP2-Rho and evaluated the nuclear extracts by flow cytometry. In this concentration range, the mean nuclear fluorescence of intact Saos-2 nuclei measured by flow cytometry correlated linearly (R²=0.84) with the intra-nuclear concentrations previously measured by FCS for all four Rho-tagged tMeCP2 proteins, regardless of overall delivery efficiency. A similar linear correlation was observed in CHO-K1 cells (vide infra). This observation suggests that flow cytometry of intact nuclei represents a rapid alternative to FCS for high-throughput analysis of nuclear delivery. It also suggests that at a treatment concentration of 2 μM, the nuclear concentration of ZF-tMeCP2-Rho was above 1.5 μM.

To better understand the relationship between overall protein uptake and nuclear delivery, we used flow cytometry to compare whole-cell fluorescence to that of intact isolated nuclei as a function of tMeCP2-Rho variant concentration and identity. In general, the fluorescence detected in the nuclei of Saos-2 cells was 2.0-5.6-fold lower than the whole-cell fluorescence, but we observed that at 2 μM some cells treated with ZF-tMeCP2-Rho and Tat-tMeCP2-Rho had nuclear fluorescent intensities as high as those of the whole cell, suggesting the majority of the protein within these cells had trafficked to the nucleus. Comparing the mean nuclear fluorescence to the mean whole-cell fluorescence, there is a general trend of dose-dependent increase in the fraction that travels to the nucleus. At treatment concentrations below 2 μM, the fraction of tMeCP2-Rho variants that reached the nucleus was low (17-30%), regardless of both concentration and whether or not tMeCP2 was fused to ZF5.3, Tat, ZF5.3(T158M). At 2 μM, however, the fraction of tMeCP2-Rho variant that reached the nucleus was significant for ZF-tMeCP2-Rho (52%), Tat-tMeCP2-Rho (51%), as well as ZF-tMeCP2(T158M)-Rho (43%).

In preparation for function studies (vide infra), we also evaluated the trafficking of each tMeCP2-Rho variant to the cytosol and nucleus of CHO-K1 cells using confocal microscopy, flow cytometry, and FCS. These results largely mirrored the results obtained in Saos-2 cells, although overall the protein concentration in the nucleus was 1.8-3.8 fold lower than observed in Saos-2 cells. Similar cell type-to-cell type variations in delivery efficiency have been observed before among HeLa cells, SK-HEP1 cells, and Saos-2 cells9,11. Nevertheless, these results provide confidence that ZF5.3 and Tat improve by 2.0-2.5 fold the nuclear delivery of tMeCP2-Rho in two model cell lines.

ZF-tMeCP2, but not Tat-tMeCP2, is Intact when it Reaches the Nucleus

Although fluorescence-based methods offer a good initial assessment of protein delivery efficiency, they must always be accompanied by biochemical studies to ensure that the fluorescent material being followed is intact9,11,42. To establish the extent to which the measured FCS values represent the concentrations of intact tMeCP2 proteins, we devised a workflow to stringently separate and isolate the cytosolic and nuclear fractions of Saos-2 cells after 1 hr treatment with 1 μM tMeCP2, ZF-tMeCP2, or Tat-tMeCP2 at 37° C. These extracts were analyzed using Western blots and an anti-Strep-tag antibody. Non-treated cells were subject to the same workflow and doped with 150 nM purified tMeCP2, ZF-tMeCP2, or Tat-tMeCP2 to calibrate the Western blots. Bands corresponding to both intact ZF-tMeCP2 and Tat-tMeCP2 are evident in the nuclear supernatant. We note that although the nuclear delivery efficiencies of ZF-tMeCP2-Rho and Tat-tMeCP2-Rho determined by FCS were comparable, Western blot analysis indicates that the concentration of intact ZF-tMeCP2 in the nucleus exceeds that of Tat-tMeCP2 by a significant margin. No band corresponding to tMeCP2 itself was observed in the nuclear supernatant, indicating that this protein was either degraded or did not enter cells at a concentration high enough to be detected. Western blot analysis of cellular extracts with antibodies recognizing highly expressed endocytic (EEA1, LAMP1, Rab7) and cytosolic (tubulin) proteins confirmed that nuclear fractions were free of detectable cytosol and endosome contaminations.

To further characterize the integrity of ZF-tMeCP2 delivered to the nucleus, we enriched the nuclear supernatant for strep-tagged proteins, treated the enriched sample with trypsin, and subjected the digest to LC-MS/MS analysis. More than 65% of the ZF-tMeCP2 sequence was detected, including fragments at the N- and C-terminus. The observation of N-terminal fragments after enrichment with a C-terminal strep tag provides further evidence that the ZF-tMeCP2 delivered to the nucleus is predominantly intact.

### ZF-tMeCP2 Accesses a HOPS-Dependent Portal for Endosomal Release

Two multisubunit tethering complexes play important roles in the endosome maturation pathway. A class C core vacuole/endosome tethering (CORVET) complex facilitates the fusion of Rab5 positive early endosomes while the fusion of Rab7 positive late endosomes to lysosomes requires the homotypic fusion and protein sorting (HOPS)-tethering complex46,47. Previous mechanistic studies indicate that efficient cytosolic and nuclear trafficking of ZF5.3 relies on the HOPS complex, but not the analogous CORVET complex10. We thus sought to investigate if this dependence also held for the ZF5.3 conjugate of tMeCP2.

We used siRNAs to knock down an essential and unique subunit of either HOPS (VPS39) or CORVET (TGFBRAP1) in Saos2 cells, using a non-targeting siRNA (RISC-free) and lipofectamine only treatment as controls. The cells were then treated with 1 μM tMeCP2-Rho, ZF-tMeCP2-Rho, or Tat-tMeCP2-Rho for 1 h and analyzed by flow cytometry and FCS. Total cellular uptake was affected minimally if at all by any gene knockdown. Knockdown of VPS39 led to a significant decrease in the concentration of ZF-tMeCP2-Rho that reached the cytosol (3.1-fold) or nucleus (4.7-fold); these fold changes are consistent with those previously observed for ZF5.3 alone10. Interestingly, knockdown of TGFBRAP1 also resulted in a significant (albeit smaller) decrease in the concentration of ZF-tMeCP2-Rho that reached the nucleus (2.4-fold). Even the trafficking of tMeCP2 itself was affected by the knockdown of both VPS39 and TGFBRAP1. It is well known that depletion of TGFBRAP1 and VPS39 can affect trafficking in a cargo-dependent manner48. The fact that delivery of tMeCP2 itself is CORVET and HOPS dependent provides one explanation for the high intrinsic permeability of this nuclear protein and deserves further study. Thus the improved nuclear delivery of ZF-tMeCP2 may result because it accesses both HOPS-dependent and CORVET-dependent portals. Notably, the only tMeCP2 conjugate whose delivery to the cytosol and nucleus was unaffected by knockdown of either VPS39 or TGFBRAP1 was Tat-tMeCP2. This result suggests that Tat-tMeCP2 gains entry into cells, albeit less efficiently, via a different subpopulation of endosomes48 or one or more non-endosomal pathways49.

Delivered ZF-tMeCP2 Interacts with Partner Proteins in the NCoR/SMRT Complex

Next we explored the function of tMeCP2 proteins delivered to the nucleus of CHO-K1 cells, which express low levels of endogenous MeCP2. If functional tMeCP2 proteins reach the nucleus, then they should interact with and sequester the essential subunits of the core NCoR/SMRT complex6, 34,36 (NCoR1, HDAC3, and TBL1/TBLR1) upon immunoprecipitation, as observed in vitro in lysates. To test this hypothesis, CHO-K1 cells were treated with 1 μM tMeCP2, ZF-tMeCP2, or Tat-tMeCP2 for 1 hr at 37° C. Nuclear proteins were rigorously isolated and incubated with streptavidin-coated magnetic beads to sequester strep-tagged tMeCP2 proteins and the proteins with which they interact. Non-treated cells were subject to the same workflow and doped with 150 nM purified tMeCP2, ZF-tMeCP2, and Tat-tMeCP2. Western blot analysis confirmed that the input nuclear fractions used for immunoprecipitation were free of detectable cytosolic (tubulin) and endosomal (EEA1, Rab7, LAMP1) contaminants and contained a higher amount of intact ZF-tMeCP2 than Tat-tMeCP2. Intact tMeCP2 could not be detected by Western blot of the nuclear extracts. Equal amounts of HDAC3 and TBLR1/TBL1XR1 were also detected in all input nuclear lysates.

Examination of the Western blots after immuno-precipitation show that both ZF-tMeCP2 and Tat-tMeCP2 sequester HDAC3 and TBLR1 from nuclear lysates in accord with their effective concentration; more ZF-tMeCP2 reaches the nucleus intact and as a result more HDAC3 and TBLR1 are sequestered. We note that the large decrease in the intensity of the Tat-tMeCP2 bands from input to pulldown emphasizes its sensitivity to degradation during the overnight incubation. The NcoR level was too low to be detected in CHO-K1 cells. We conclude that ZF-tMeCP2 enters the cell nucleus and interacts more productively with partner proteins than either tMeCP2 or Tat-tMeCP2.

Delivered ZF-tMeCP2 Selectively Represses Transcription from Methylated DNA

In the nucleus, MeCP2 acts as a bridge to deliver the NCoR/SMRT complex to methylated promoters; this recruitment represses transcription23. We devised a flow cytometry assay to evaluate whether delivered tMeCP2 variants that reach the nucleus preferentially repress transcription of methylated over non-methylated reporter genes. Briefly, CHO-K1 cells were transfected with a methylated or non-methylated plasmid encoding mNeonGreen fluorescent protein under the control of the small nuclear ribonucleoprotein polypeptide N (SNRPN) promoter. MeCP2 binds to the methylated form of SNRPN to downregulate downstream genes50, 51. A short signal sequence (PEST) was encoded at the C-terminus of mNeonGreen to promote its turnover and improve assay sensitivity52. Functional tMeCP2 variants that reach the nucleus should selectively repress transcription from cells transfected with the methylated SNRPN promoter, leading to less mNeonGreen fluorescence relative to controls. By contrast, delivery of a trace, non-functional, or nonspecific tMeCP2 variant will either not repress transcription or do so without selectivity for the methylated promoter.

To evaluate mNeonGreen expression, CHO-K1 cells transfected with methylated or non-methylated plasmid DNA were treated for 1 hr at 37° C. with 1-5 μM tMeCP2, ZF-tMeCP2, Tat-tMeCP2 or ZF-tMeCP2(T158M) and the fluorescence emission at 530±30 nm was monitored using flow cytometry. Cells treated with tMeCP2 itself displayed high mNeonGreen fluorescence levels regardless of promoter methylation state. By contrast, cells treated with ZF-tMeCP2, Tat-tMeCP2, as well as ZF-tMeCP2(T158M) all showed dose-dependent decreases in mNeonGreen fluorescence. The highest levels of methylation-specific transcriptional repression was observed in cells treated with 5 μM ZF-tMeCP2, Tat-tMeCP2, as well as ZF-tMeCP2(T158M), although measurable effects were seen at concentrations as low as 2 p M. In general, the levels of transcriptional repression were lower in cells treated with Tat-tMeCP2.

These data are in line with the protein nuclear concentrations and diffusion time and DNA binding kinetics measured by FCS. FCS is useful not only for measuring the concentration of a fluorescently tagged protein or macromolecule within the cytosol or nucleus, but also for determining its intracellular diffusion time (τ_{diff})^{42,43}. Fitting the autocorrelation curves obtained from intra-nuclear measurements in CHO-K1 cells with a two-component 3D diffusion equation53 identified a population of tMeCP2-Rho variants that diffuses freely in the nucleoplasm (fast fraction, F_{fast}) and a second population that diffuses slowly (slow fraction, F_{slow}), presumably because it is bound to DNA. At 1 μM, the fraction of ZF-tMeCP2 diffuses slowly (F_{slow}=26.3%) is higher than that of ZF-tMeCP2(T158M) (16.3%) in accord with relative methylated DNA affinities determined in vitro and in cells33. Thus the higher nuclear concentration of ZF-tMeCP2(T158M) is counterbalanced by the low DNA binding population; the result is no significant transcriptional repression. At 2 μM, ZF-tMeCP2, Tat-tMeCP2, as well as ZF-tMeCP2(T158M) all reached the nucleus at significantly higher levels than tMeCP2 and show higher levels of DNA binding, with values of F_{slow} of 24.9%, 33.7% and 25.6%, respectively; the result is observable transcriptional repression.

Differences in transcriptional repression are most apparent when the ratio of mNeonGreen expression in cells transfected with methylated vs. non-methylated promoters are compared. As expected, no selective repression is observed in cells treated with tMeCP2. Tat-tMeCP2 treatment led to higher levels of mNeonGreen repression in cells transfected with a non-methylated promoter, as suggested by the in vitro DNA binding results; Tat alone possesses high non-specific DNA binding affinity (K_{d}=126 nM)⁵⁴. The highest levels of methylation-dependent transcriptional repression were observed in cells treated with ZF-tMeCP2 and ZF-tMeCP2(T158M). As a previous study suggested, MeCP2(T158M) is capable of binding to methylated DNA in a protein level-dependent manner55. At low treatment concentrations (1-2 μM), most ZF-tMeCP2(T158M) was sequestered in the nucleolus, so the effective nuclear concentration of ZF-tMeCP2(T158M) is not high enough to rescue its reduced methylated DNA binding affinity. As more protein reaches the nucleus at 5 μM treatment, a higher level of the methylated promoter is bound and repression is observed. Taken together, these data indicate that the fusion of ZF5.3 to tMeCP2 does not interfere with the protein's selectivity towards methylated DNA. ZF-tMeCP2 reaches the nucleus at a concentration high enough to observe methylation-dependent transcription repression. Notably, although fluorescent detection implies relatively comparable nuclear delivery of ZF-tMeCP2 and Tat-tMeCP2, the latter is largely degraded, impairing its ability to regulate downstream transcriptions.

### Discussion

Efficient protein delivery remains a significant and unmet challenge in an era exploding with novel protein-based therapeutic strategies. Almost all FDA-approved biologics are delivered via injection, and operate within serum, on cell surfaces, or within endosomal vesicles. The impact of protein-based strategies would expand exponentially if proteins could reliably evade endosomal degradation, escape endosomal vesicles, and remain functional. Yet progress towards this goal has been exceptionally slow. Most delivery strategies are inefficient9, rely on degradation-prone molecular scaffolds26,29, operate via undefined or multifarious mechanisms56, and detect activity using amplified or indirect assays9,57.

In this work, we address these challenges by using the stable mini-protein ZF5.3 to hijack the endosomal maturation machinery and guide the methyl-CpG-binding-protein 2 (MeCP2) into the nucleus at a therapeutically relevant concentration. Rigorous analyses verify the absence of significant nuclear degradation and the presence of MeCP2-specific activity. Multiple independent approaches, including intact nuclear flow (INF), a novel and general application of flow cytometry, quantitatively assess delivery in a non-amplified and high-throughput manner. Finally, the remarkable level of nuclear delivery by ZF-tMeCP2 enables its potential application to reverse Rett syndrome phenotypes; a modest dose to Saos-2 cells (1 h, 1 μM) results in a nuclear concentration of 2.1×10⁵ molecules/cell, which is within the range of endogenous MeCP2 in HEK29324 and NeuN positive neurons from mature mouse brain1 (1.6×10⁵ and 160×10⁵ molecules/cell, respectively). This delivered concentration is two orders of magnitude higher than a previously reported Tat-MeCP2 conjugate that could rescue some RTT-related symptoms26, 27, 29.

### Methods and Materials

### Plasmid Construction

We initially attempted to express and purify full-length MeCP2 as a C-terminal fusion with ZF5.3 in Escherichia coli, but the yield was low and could not be optimized. The DNA sequence for tMeCP2(ΔNC) was obtained by deleting the N-terminal (residues 13-71) and the C-terminal (residues 313-484) sequences of mouse tMeCP2_e2 (protein sequence identifier: Q9Z2D6-1). The codon-optimized gblock encoding ZF5.3-tMeCP2(ΔNC)-LPETGG-Strep was purchased from Integrated DNA Technologies (IDT). A GSG linker was added between ZF5.3 and tMeCP2(ΔNC) and a GSSGSSG linker was inserted before the C-terminal LPETGG-Strep tag. A pET His6 MBP TEV LIC cloning vector (Addgene Plasmid #29656) was digested using restriction enzymes XbaI and BamHI to remove the His6-MBP-TEV portion and the synthetic gblocks containing complementary overhangs were incorporated into the pET vector using Gibson assembly. To generate tMeCP2(ΔNC)-LPETGG-Strep or Tat-tMeCP2(ΔNC)-LPETGG-Strep, the ZF5.3-tMeCP2(ΔNC)-LPETGG-Strep plasmid was double digested with XbaI and SacII respectively to remove the ZF5.3 segment, and ligated back using Gibson assembly with double-stranded fragments containing corresponding sequences. Point mutations (T158M, P302L) in the protein were generated by Q5 site-directed mutagenesis. The identity of all plasmids were confirmed by Sanger and whole plasmid sequencing. Relevant DNA and protein sequences are listed in Table 1

### Protein Expression and Purification

All tMeCP2 variants (with and without labeled fluorophore) are purified as described in

### Supplementary Methods 1.

### Circular Dichroism

Circular Dichroism measurements were performed following a previously published protocol.60 Wavelength-dependent circular dichroism spectra were recorded using an AVIV Biomedical, Inc. (Lakewood, NJ) Circular Dichroism Spectrometer Model 410 at 25° C. in a 0.1 cm pathlength quartz cuvette. CD spectra were collected at ˜12 μM protein concentration in 20 mM HEPES, 300 mM NaCl, 10% glycerol pH 7.6 between 300 and 200 nm at 1 nm intervals with an averaging time of 5 seconds. A separate wavelength spectrum for storage buffer alone was performed to verify no interfering buffer signal. Raw ellipticity values were converted to mean residue ellipticity. The protein secondary structure was predicted using the webserver BeStSel.

### Fluorescence Polarization

Samples for fluorescence polarization (FP) studies were prepared by mixing assay buffer (25 mM Tris-HCl, 6% glycerol, 100 μg/mL BSA, 0.1 mM EDTA, and 0.1 mM TCEP, 250 mM KCl, pH 7.6), different tMeCP2 variants with final concentrations ranging from 1 nM to 5 μM and 20 nM DNA probe. The DNA probes carrying an N terminal 6-fluorescein61 (methylated or non-methylated, Table 1) were synthesized by annealing equimolar of complementary single strands derived from a 22 bp DNA sengment of mouse BDNF promoter IV (purchased from IDT) at 95° C. for 2 min, followed by 60° C. for 10 min and cooled to 4° C.62. The samples were incubated for 30 min at room temperature to reach equilibrium and added to a 96 well half area solid black plate (Corning #3993, 50 μL per well).

FP measurements were performed using a SpectraMax M5 plate reader with an excitation wavelength at 480 nm and an emission window of 515 to 520 nm. Measurements from three wells were averaged for each determination. To calculate K_{D}, the data were fitted by least squares regression to an equilibrium binding equation based on the Langmuir model with modifications explicitly considering the total DNA concentration39 using GraphPad Prism 9. While previous studies reported both MBD39 and full-length tMeCP238,63 can bind cooperatively to DNA, the high salt concentration used in the assay buffer suppressed binding cooperativity and preserved the specificity for methylated DNA63. The Hill coefficients fitted for all conditions in this experiment were close to 1.

### Cell Culture

Saos-2 cell stock was purchased from the American Type Culture Collection (ATCC). NIH3T3, HeLa and CHO-K1 cell stocks were purchased from UC Berkeley Cell Culture Facility. Saos-2 cells were cultured in McCoy's 5A medium (Hyclone) with phenol red containing 15% fetal bovine serum (FBS), penicillin and streptomycin (P/S, 100 units/mL and 100 g/mL respectively), 1 mM sodium pyruvate (Gibco), 2 mM GlutaMax (Gibco). NIH3T3 and HeLa cells were cultured in Dulbecco's Modified Eagle's Medium (DMEM, Gibco) with 10% FBS and P/S. CHO-K1 cells were cultured in F12 Nutrient Mixture (Ham) media (Gibco) with L-Glutamine, 10% FBS and P/S. All cell cultures were incubated at 37° C., in 5% CO₂.

### In Vitro Pull-Down Assay

Nuclear lysate of NIH3T3 cells were obtained as described in Supplementary Methods 2. 200 μL of the nuclear lysate was mixed with 1.5 μM of purified tMeCP2 variants overnight at 4° C. in the coIP buffer36 (20 mM HEPES, pH 7.6, 10 mM KCl, 150 mM NaCl, I mM MgCl₂, 0.1% Triton X-100 (vol/vol), protease inhibitors (Roche), 15 mM BM E). On the next day, to pull down tMeCP2 protein complexes. MagStrep “type3” XT magnetic beads (IBA 2-4090-002) following the manufacturer's note were used. The beads were pre-equilibrated with colP buffer and incubated with the mixture of nuclear lysate and tMeCP2 for 45 min at RT on a rotating wheel. After separating the beads from the unbound supernatant using a magnetic separator, the beads were washed with 200 μL colP buffer two times (quickly vortex and centrifuge). The pul-down proteins were eluted with 10 μL 5×SDS-PAGE loading dye and 10 μL milliQ water at 95° C. for 3 min. The input and pulldown samples were analyzed by western blot using primary antibodies against Strep-tagII (IBA 2-1509-001), HDAC3 (CST 85057S), TBLR1/TBL1XR1 (Abcarn ab190796), NCoR1 (CST 5948S), and secondary antibody IRP-linked Anti-rabbit IgG (CST 7074S).

### Fluorescence-Based Assay for Protein Delivery

The fluorescence-based experiments (confocal microscopy, fluorescence correlation spectroscopy and whole-cell flow cytometry) to evaluate protein delivery were performed according to previously published protocols42. For delivery into Saos-2 cells, 500 μL tMeCP2 variants diluted in McCoy's 5A medium (—FBS, -phenol red) to final concentrations (0.5-2 μM) were added to the cells plated in a 6-well plate to incubate for 1 h at 37° C., 5% CO₂. 300 nM of the nuclear stain Hoechst 33342 was added to cells 5 min prior to the end of incubation time.

For experiments using CHO-K1 cells, a similar protocol was followed except: the cells were plated in DMEM media supplemented with 1× non-canonical amino acid (+10% FBS, -phenol red) on day 1. On day 2, 1-5 μM tMeCP2 variants were diluted in DMEM media supplemented with 1×non-canonical amino acid (-phenol red) for delivery. DMEM media supplemented with 1× non-canonical amino acid (+10% FBS, -phenol red) was used to quench the trypsin reaction and rinse the 6-well plate. Cells were resuspended in DMEM media supplemented with 1× non-canonical amino acid (-phenol red) for confocal microscopy and fluorescence correlation spectroscopy studies.

### Confocal Microscopy and Fluorescence Correlation Spectroscopy (FCS)

Confocal microscopy and FCS experiments were performed using a STELLARIS 8 confocal microscope (Leica) with a Hybrid HyD×detector, and an HC PL APO 63x/1,20 W motCORR CS2 water (63 W) immersion objective (Leica). The correction collar of the objective was adjusted based on the thickness of an 8-well chambered coverglass prior to experiments. The fluorophore was excited at 561 nm and the fluorescence filter was set to 570-660 nm. The pinhole of the 561 nm laser was set to 1 AU. At the beginning of the experiments, the imaging chamber was equilibrated to 37° C., 5% CO₂. Before each FCS measurement, a confocal image of the cells was obtained and the laser was directed to either the cytosol or nucleus of the cells to obtain FCS data in discrete cellular locations. Areas around the punctate fluorescence of endosomes were avoided. All FCS measurements were collected in ten consecutive five-second time intervals. A minimum of 20 cells was measured per replicate, and data from at least two biological replicates were collected for each condition. To measure the diffusion coefficients of tMeCP2-Rho variants in vitro, proteins were diluted in DMEM (25 mM HEPES, -phenol red) to 100 nM fluorophore concentration, and their autocorrelation data were collected at 37° C. (10 repeats, 5 s intervals). Because some phototoxicity was observed during FCS in CHO-K1 cells, besides keeping the cells at 37° C., 5% CO, we avoided shining light on a single cell two times by measuring the cytosol, and nuclear concentrations in separate cells.

The cytosolic and nuclear concentrations of fluorescently tagged tMeCP2-Rho variants in cells were calculated by fitting the measured data to a 3D anomalous diffusion equation (cytosol) and a two-component 3D diffusion equation (nucleus). We further adjusted the calculated concentrations based on the fluorophore labeling efficiency of each protein to obtain the true intracellular concentrations.

### Flow Cytometry

Flow cytometry measurements were performed at room temperature with an Attune NxT flow cytometer. The fluorophore Rho was excited with a laser at 561 nm, and the emission filter was set at 585±16 nm. For whole-cell flow cytometry, at least 10,000 cells were analyzed for each sample and at least three biological replicates were measured for each condition. To isolate the intact nuclei, cells treated with tMeCP2 variants were washed and trypsinized as described above. After centrifugation at 200 g for 3 min, the pellet was resuspended in 1 mL DPBS and 200 μL was saved for whole-cell flow cytometry analysis. The rest of 800 μL was pelleted at 200 g for 3 min, resuspended with precooled 1 mL isotonic sucrose buffer (290 mM sucrose, 10 mM imidazole, pH 7.0, 1 mM DTT, and 1 cOmplete protease inhibitor cocktail (Roche) per 10 mL buffer) and transferred to a 1.5 mL microcentrifuge tube. After centrifuging at 10,000 g, 4° C. for 1 min, the pellet was resuspended in 200 μL of isotonic sucrose buffer+0.1% NP-40 and centrifuged at 1000 g, 4° C. for 10 min. The resulting intact nuclei pellet was resuspended in 200 μL PBS. 20 μL sample was mixed with Trypan blue to check the intactness of the isolated nuclei under an inverted microscope while the rest was analyzed by flow cytometry. The collected data were analyzed using FlowJo software (version 10.8.1, FlowJo, LLC).

### RNAi

siRNA-mediated knockdown was performed as described previously10 using Lipofectamine RNAiMAX transfection reagent (Invitrogen) and siRNA (100 nM, Dharmacon). All siRNA used was listed in Table 1. Saos-2 cells were transfected for 4 hours and grew in McCoy's 5A medium (2 mL/well, 15% FBS, 1 mM sodium pyruvate, 2 mM GlutaMax, -phenol red, -P/S) for 72 h at 37° C., 5% CO2 for optimal gene knockdown. The effect of gene knockdown was studied using flow cytometry and FCS as described above.

### RT-qPCR

The degree of siRNA-mediated gene knockdown after 72 h was evaluated using RT-qPCR as described previously10. For RT-qPCR, cDNA was amplified using 150 nM gene-specific primers (IDT, PrimeTime qPCR primers) with SsoFast EvaGreen Supermix (Bio-Rad) on a Bio-Rad CFX96 real-time PCR detection system. Three biological replicates were evaluated. Each run includes the siRNA-targeted gene, GAPDH as the reference gene and their respective negative controls (no reverse transcriptase and no template). Each sample was run in triplicate. Primers used for reverse transcription into cDNA and qPCR were listed in Table 1.

### Cytosolic Fractionation to Determine Protein Intactness

The cellular fractionation experiment was adapted from previous reports9-11 with modifications to ensure pure nuclei isolation. Saos-2 cells were grown to ˜2.5×10⁶ cells in a 100 mm dish in McCoy's 5A medium (+15% FBS, -phenol red). For protein delivery, tMeCP2-Strep variants diluted in clear McCoy's media without FBS to final concentration of 1 μM were incubated with cells at 37° C. with 5% CO₂ for 1 h. One dish of cells with the same clear McCoy's media added was included as a non-treated control. After incubation, cells were lifted, washed, and lysed as previously described.11 The homogenized cell lysate was centrifuged for 10 min at 800 g, 4° C. The crude supernatant was re-centrifuged at 1200 g, 4° C. for 10 min. The resulting supernatant was transferred to a polycarbonate ultracentrifuge tube and centrifuged at 350 kg for 30 min (TL-100; Beckman Coulter, TLA-100 rotor (20×0.2 mL) to isolate the cytosolic fraction. The crude nuclei pellet was washed in 500 μL of isotonic sucrose buffer (290 mM sucrose, 10 mM imidazole, pH 7.0, 1 mM DTT, and 1 cOmplete protease inhibitor cocktail (Roche)) supplemented with 0.15% NP-40. After incubating on ice for 10 min, the mixture was centrifuged at 1200 g, 4° C. for 10 min. The resulting pure nuclear pellet was first resuspended with 20 μL milliQ water and 2 μL benzonase (Sigma, 71206) at room temperature for 20 min. 80 μL high salt extraction buffer64 (20 mM H EPES, pH 7.6, 1.5 mM MgCl₂, 420 mM NaCl, 0.2 mM EDTA, 25% glycerol, protease inhibitor (Roche)) was then added to the mixture and vortexed vigorously during the 30 min incubation period on ice. The extracted nuclear supernatant was obtained by centrifuging at 21,000 g, 4° C. for 5 min. For SDS-PAGE gel, both the cytosolic pellet and the nuclear pellet were dissolved in 20 μL of 5×SDS gel loading dye and boiled at 95° C. for 5 min. All the supernatant samples (cytosolic, wash, nuclear) were prepared by mixing 20 μL sample with 5 μL 5×SDS gel loading dye and boiled at 95° C. for 5 min. Loading controls were generated by adding 150 nM of purified tMeCP2-Strep variants to non-treated nuclear supernatant. Western blot analysis was performed using primary antibodies against Strep-tagII (IBA 21-509-001), MeCP2 (CST 34565), LAMP 1 (CST 90915), EEA1 (CST 32885), tubulin (CST 21255), Rab 7 (CST 9367S) and the secondary antibody HRP-linked Anti-rabbit IgG (CST 7074S).

### LC-MS/MS Analysis

1 μM ZF-tMeCP2-Strep was incubated with Saos-2 cells (37° C., 5% CO₂, 1 h) and the nuclear supernatant was isolated as described in the section above. ZF-tMeCP2-Strep delivered to the nucleus was enriched by incubating the nuclear supernatant with MagStrep “type3” XT magnetic beads (IBA 2-4090-002) for 45 min at RT in a rotating wheel. After separating the beads from the unbound supernatant using a magnetic separator, the pull-down proteins were eluted with 10 μL 5×SDS-PAGE loading dye (diluted to 2.5× by 10 μL milliQ water) at 95° C. for 3 min. The sample was run on a 10% SDS-PAGE gel (Biorad, Mini-PROTEAN® TGX™) and visualized by Gelcode Blue® Coomassie stain (Pierce) following the manufacturer's note. The band corresponding to ZF-tMeCP2-Strep was extracted from the gel and digested with trypsin, and the resulting peptides were dried and resuspended in buffer: 5% acetonitrile/0.02% heptaflurobutyric acid (HBFA)). Mass spectrometry was performed by the Proteomics/Mass Spectrometry Laboratory at UC Berkeley as described in Supplementary Methods 3.

### Determining Endogenous Level of MeCP2 and NCoR

1×10⁶ of Saos-2, CHO-K1, NIH3T3 or HeLa cells were lysed with 100 μL M-PER™ Mammalian Protein Extraction Reagent (Thermo Scientific, 78501) for 10 min following the manufacturer's instructions. The lysates were centrifuged at 14,000 g for 15 min. For SDS-PAGE gel, the pellets were dissolved in 20 μL of 8 M urea and 5 μL 5×SDS gel loading dye and boiled at 95° C. for 5 min. The supernatant samples were prepared by mixing 20 μL sample with 5 μL 5×SDS gel loading dye and boiled at 95° C. for 5 min. Western blot analysis was performed using primary antibodies against MeCP2 (34565), NCoRl (CST 5948S), GAPDH (CST 2118S), tubulin (CST 21255) and the secondary antibody HRP-linked Anti-rabbit IgG (CST 7074S).

### In Cellulo Coimmunoprecipitation Assay

On day 1, ˜5×10⁶ CHO-K1 cells were plated in 150 mm dishes in F12 Nutrient Mixture (Ham) media with L-Glutamine (+10% FBS). On day 2, for protein delivery, cells were washed with 25 mL DPBS three times and treated with 1 μM tMeCP2-Strep variants (WT, ZF or Tat, diluted in F12 media without FBS) at 37° C. with 5% CO₂ for 1 h. Three dishes of cells were incubated with the same F12 media added as controls. After incubation, cells were lifted, washed and lysed as described in the cellular fractionation section. For lysis, the cells from each dish were then suspended in 300 μL of isotonic sucrose buffer (290 mM sucrose, 10 mM imidazole, pH 7.0, 1 mM DTT, and 1 cOmplete protease inhibitor cocktail (Roche)), transferred to 0.5 mL microtubes containing 1.4 mm ceramic beads (Omni International) and homogenized using a Bead Ruptor 4 (Omni International) for 10 s at speed 1. The homogenized cell lysate was centrifuged for 10 min at 800 g, 4° C. The supernatant contained cytosolic proteins and the crude nuclei pellet was washed in 500 μL of isotonic sucrose buffer supplemented with 0.15% NP-40. After incubating on ice for 10 min, the mixture was centrifuged at 1200 g, 4° C. for 10 min. After separating the wash 1 supernatant, the pellet was washed again with 400 μL of isotonic sucrose buffer supplemented with 0.15% NP-40 and centrifuged at 1200 g, 4° C. for 10 min. The resulting pure nuclear pellet was first resuspended with 70 μL low salt buffer (20 mM HEPES, pH 7.6. 1.5 mM MgCl₂, 10 mM KCl, 25% glycerol, protease inhibitor (Roche)) and 2.5 μL benzonase (Sigma, 71206), transferred to 0.5 mL microtubes containing 1.4 mm ceramic beads and homogenized using a Bead Ruptor 4 for 10 s at speed 5 for three times (30 s interval on ice between each session). The homogenized nuclear lysate was then incubated at 37° C. for 10 min on a rotating wheel for benzonase digestion. 1M NaCl solution was added to the lysate to a final NaCl concentration of 430 mM. The mixture was homogenized again for 10 s at speed 5 and incubated for 1 h at 4° C. on a rotating wheel. The extracted nuclear supernatant was obtained by centrifuging at 18,000 g, 4° C. for 10 min.

For two control samples, 150 nM purified ZF- or Tat-tMeCP2-Strep was doped in nuclear supernatant. The input samples were prepared by taking 12.5 μL of nuclear supernatant and mixing with 7.5 μL milliQ water and 5 μL 5×SDS gel loading dye. The rest of the nuclear supernatant was diluted to 150 mM NaCl with low salt buffer supplemented with 15 mM BME and incubated with MagStrep “type3” XT magnetic beads (IBA 2-4090-002) overnight at 4° C. on a rotating wheel. On the next day, the unbound supernatant was separated from the beads using a magnetic separator. The beads were washed with 200 μL coIP buffer two times (quickly vortex and centrifuge) and eluted with 10 μL 5×SDS-PAGE loading dye (diluted to 2.5× by 10 μL milliQ water) at 95° C. for 3 min. The input and pulldown samples were analyzed by western blot using primary antibodies against Strep-tagII (fBA 2-1509-001), HDAC3 (CST 85057S), TBLR1/TBL1XR1 (CST 74499S), and secondary antibody HRP-linked Anti-rabbit IgG (CST 7074S). To detect for contamination from other cellular compartments, the input and pulldown samples, as well as other fractions during the isolation process (cytosolic supernatant, wash 1, and wash 2), were analyzed by western blot using primary antibodies against EEA1 (CST 3288S), tubulin (CST 2125S), Rab 7 (CST 9367S) and the secondary antibody HRP-linked Anti-rabbit IgG (CST 7074S).

### In Cellulo Transcription Assay

To construct the reporter plasmid, the mNeonGreen gene was amplified from a pCS2+mNeonGreen-C Cloning Vector (Addgene #248605) using design primers (Table 1). The SNRPN promoter was amplified from mouse genomic DNA (Promega, G3091) using design primers (Table 1). The amplified SNRPN promoter segment was incorporated into a mammalian expression vector pNL1.2 (Promega, N1011) using the two restriction sites NheI and HindIII followed by gibson assembly. The assembled pNL1.2-SNRPN plasmid was confirmed by Sanger sequencing and digested again with HindIlI and EcoRI to incorporate the amplified mNeonGreen gene downstream of SNRPN using a gibson assembly kit. The final assembled plasmid was named as pSNRPN-mNeonGreen-PEST. To generate methylated promoter, the plasmid was with HpaII methyltransferase (NEB, M0214S) following manufacturer's instructions. Complete methylation was checked by testing the plasmid's resistance to HpaII restriction enzyme digestion.

To perform the assay, on day 1 CHO-K1 cells grown to ˜70% confluency in 60 mm dishes were transfected with either methylated or non-methyalted reporter plasmids in reduced serum media (OPTI-MEM, Gibco, 31985-062) using a TransIT CHO-K1 transfection kit (Mirusb Bio) following manufacturer's instructions. After 12 h the cells were washed with DPBS two times and lifted with enzyme-free cell dissociation buffer (Gibco). The lifted cells were pelleted, counted and plated into a 24-well plate in 1.2×10⁵ cells/well in growth media (F12 Nutrient Mixture (Ham) media (Gibco) with L-Glutamine+10% FBS). On day 2, the medium was aspirated and the cells were washed with 1 mL DPBS two times. Then 130 μL of 1-5 μM tMeCP2-Strep variants diluted in F12 medium (—FBS, -P/S) were added to the cells and incubated for 1 h at 37° C., 5% CO₂. After delivery, cells were washed with 1 mL of DBPS three times and the media was replaced with the growth media for a one-hour incubation. After incubation, the cells were lifted with 200 μL TrypinLE Express for 5 min at 37° C., 5% CO₂, quenched with 1 mL growth media, and transferred to 1.5 mL microcentrifuge tubes. The cells were pelleted by centrifugation at 500 g for 3 min, and washed with 1 mL clear DMEM medium. After centrifuging at 500 g for 3 min, the pellets were resuspended in 200 μL clear DMEM medium and analyzed using an Attune NxT flow cytometer (Life Technologies).

For flow cytometry, the mNeonGreen protein was excited with a laser at 488 nm, and the emission filter was set at 530±30 nm. 130 μL (at least 50,000 cells) were analyzed for each sample and at least three technical and biological replicates were measured for each condition. Cells having green fluorescence were gated based on the background fluorescence level of cells not transfected with reporter plasmids.

### Supplementary Methods 1. Protein Expression and Purification

The plasmids encoding tMeCP2 variants were each transformed into E. coli BL21-CodonPlus(DE3)-RP cells (Agilent, #230255) and selected on a kanamycin (Kan)-chloramphenicol (Cm)-double resistant LB agar plate. For protein expression, a starter culture was generated by inoculating a colony in 25 mL of LB media with 25 μL each 1000×Kan and Cm stock solutions. After overnight incubation at 37° C. with shaking at 200 rpm, 20 mL starter culture was transferred into 2 L of LB with Kan and Cm. The culture was grown at 37° C. until OD₆₀₀=0.6. At this point, 1 mM IPTG was added to induce expression, and growth continued at 30° C. for 3 h. The cells were pelleted by centrifugation at 4,300 g for 40 min and pellets resuspended in 20 mM HEPES (pH 7.6), 200 mM NaCl and 0.1% Nonidet P-40, with a tablet of cOmplete, Mini EDTA-free protease inhibitor cocktail (Roche). After lysis by homogenization, the cell lysate was centrifuged at 10,000 g for 30 min. The resulting clear lysate was passed through a 0.22 μm hydrophilic PVDF membrane filter (EMD Millipore) and subjected to a two-step purification using fast protein liquid chromatography (ÄKTA pure). First, the clear lysate was loaded onto a 5 mL HiTrap® SP HP column (Cytiva), and eluted with buffer containing 20 mM HEPES (pH 7.6), 10% glycerol, with a linear gradient from 200 mM NaCl to 1 M NaCl over 20 column volumes (CV). The fractions containing the tMeCP2 variant of interest were pooled and further purified with a 5 mL StrepTrap HP column (Cytiva). The column was washed with 5CV of wash buffer (20 mM HEPES (pH7.6), 500 mM NaCl, 10% glycerol) and eluted with wash buffer supplemented with 2.5 mM d-Desthiobiotin. The eluted desired fractions were combined, concentrated, and buffer exchanged into the final storage buffer (20 mM HEPES (pH7.6), 300 mM NaCl, 10% glycerol) using a PD-10 desalting column (Cytiva). For ZF5.3 tagged proteins, the storage buffer was supplemented with 100 μM ZnCl₂ and 1 mM dithiothreitol (DTT). After each column chromatography, the elution fractions were visualized by SDS-PAGE. The final purified recombinant tMeCP2 proteins were analyzed by mass spectrometry (Agilent 6530 QTOF LCMS) and western blot (anti-Strep-tag II antibody conjugated to horseradish peroxidase, IBA 2-1509-001) and stored at −80° C. until needed. Protein concentrations were calculated based on the Beer-Lambert law A=εbc, where A is the absorbance at 280 nm measured on a nanodrop spectrophotometer (ND-1000), ε is the extinction coefficient of the protein, and b is the optical path length (1 cm).

### Sortase-Mediated Conjugation of Fluorophore to Proteins

The synthesis and purification of GGGK-Lissamine rhodamine B (Rho) were performed as previously described10. Before use in sortase-mediated labeling experiments, the peptide was resuspended in the minimum quantity of DMSO. The concentration of the solution was calculated based on the Beer-Lambert law A=εbc, as described above.

Two variants of the hepta-mutant Staphylococcus aureus Sortase A (SrtA7m) were used to conjugate rhodamine to ZF5.3 tagged- and non-ZF5.3 tagged-tMeCP2 proteins due to different requirements in the downstream purification process after labeling. Two plasmids, one encoding StrepTagII-SrtA7m-His6, the other His6-SUMO-SrtA7m, were transformed into E. Coli BL21(DE3) Gold cells (Agilent, #230132) and selected against appropriate bacterial resistance (kanamycin for StrepTagll-SrtA7m-His6 and carbenicillin for His6-SUMO-SrtA7m). Both proteins were purified as described previously11 with slight modification. After expression, the cell pellet was resuspended in lysis buffer (20 mM HEPES (pH 7.6), 150 mM NaCl, 10% glycerol, and a tablet of cOmplete, Mini EDTA-free protease inhibitor cocktail (Roche)). After sonication, the clear lysate was incubated with 1.5 mL of TALON@metal affinity resin (Takara) pre-equilibrated with lysis buffer for 1 hr at 4° C. The mixture was transferred to a 15 mL disposable column, and washed with 45 mL of wash buffer (20 mM HEPES (pH 7.6), 500 mM NaCl, 10% glycerol, 10 mM imidazole). The proteins were eluted in 15 mL of elution buffer (20 mM HEPES (pH 7.6), 150 mM NaCl, 10% glycerol, 200 mM imidazole), and analyzed on an SDS-PAGE gel. The desired fractions were dialyzed into 20 mM HEPES (pH7.6), 300 mM NaCl, 10% glycerol overnight at 4° C. and stored at −80° C. until needed. Protein concentrations were determined by the PierceT^{M} 660 nm protein assay (ThermoFisher, 22660), using bovine serum albumin (BSA) as a standard.

For His6-SUMO-SrtA7m, the His6-SUMO tag was subsequently cleaved by incubating the protein with His6-SUMO protease purified as described previously11 (2:1 molar ratio) at RT for 2 h. After incubating the reaction mixture with TALON® metal affinity resin for 1 hr at 4° C., the cleaved SrtA7m protein was isolated from the His-tagged impurities in the flow-through and wash fractions. The desired fractions determined by SDS-PAGE gel were dialyzed into 20 mM HEPES (pH 7.6), 300 mM NaCl, 10% glycerol overnight at 4° C., and stored at −80° C. until needed. Protein concentrations were determined by the Pierce™ 660 nm protein assay using BSA as a standard.

To generate fluorescently tagged proteins, 35 μM ZF5.3 tagged tMeCP2-LPETGG-Strep (ZF or T158M) were incubated with 75 μM SrtA7m and 200 μM GGGK-Lissamine rhodamine B (Rho) for 1 hr at 37° C., in the reaction buffer 20 mM HEPES (pH 7.6), 300 mM NaCl, 10% glycerol. To isolate ZF5.3-tMeCP2-Rho/ZF5.3-tMeCP2-T158M-Rho, the reaction mixtures were incubated with 1 mL of TALON@metal affinity resin pre-equilibrated with reaction buffer for 1 hr at 4° C. and transferred to a 15 mL disposable column. The column was washed with 30 mL of reaction buffer. The ZF5.3 fusion proteins binding to the TALON resin were eluted with 10 mL elution buffer (reaction buffer with 250 mM imidazole) and analyzed on an SDS-PAGE gel. The absence of unconjugated Rho in the purified protein was confirmed by fluorescence imaging. The desired fractions were combined and dialyzed into 20 mM HEPES (pH 7.6), 300 mM NaCl, 10% glycerol, 100 μM ZnCl₂ and 1 mM DTT overnight at 4° C.

For fluorophore conjugation of non-ZF5.3-tagged tMeCP2, 35 μM tMeCP2-LPETGG-Strep or Tat-tMeCP2-LPETGG-Strep were incubated with 75 μM StrepTagII-SrtA7m-His6 and 200 μM GGGK-Rho for 1 hr at 37° C., in the reaction buffer 20 mM HEPES (pH 7.6), 300 mM NaCl, 10% glycerol. After incubation with 1 mL of TALON@metal affinity resin pre-equilibrated with the reaction buffer for 1 hr at 4° C., the reaction mixture was loaded to a 15 mL disposable column. The proteins tMeCP2-Rho and Tat-tMeCP2-Rho were collected during the wash step with 20 mL of reaction buffer. After visualization on an SDS-PAGE gel, the desired protein fractions were concentrated to 2.5 mL using a 3 k MWCO centrifugal filter unit (Amicon®) and passed through a PD-10 desalting column (Cytiva) to remove unreacted free Rho in the solution.

The final purified proteins were analyzed by SDS-PAGE gel and mass spectrometry (Agilent Agilent 6530 QTOF LCMS) and stored at −80° C. The total protein concentrations were determined using the Pierce™ 660 nm protein assay and a standard curve generated by purified tMeCP2-LPETGG-Strep variants of known concentrations. The concentration of fluorophore-conjugated proteins was inferred from the Rho concentration, which was calculated based on the Beer-Lambert law A=εbc, where A is the absorbance at 570 nm measured on a Nanodrop spectrophotometer, ε is the extinction coefficient of Rho in water (112000 M⁻¹ cm⁻¹). The efficiency of Rho conjugation to tMeCP2 was calculated by dividing the Rho concentration by the total protein concentration.

### Supplementary Methods 2. NIH3T3 Nuclear Lysate Isolation

To obtain the nuclear lysate, the intact nuclei were first prepared based on a protocol outlined by Millipore Sigma (https://www.sigmaaldrich.com/US/en/technical-documents/protocol/protein-biology/protein-lysis-and-extraction/extraction-from-tissue) with slight modifications. Four 150-mm dishes of NIH3T3 cells were grown to 80% confluency (˜5.5 million per plate). After washing twice with DPBS, the cells were lifted using 8 mL of enzyme-free cell dissociation buffer (Gibco) for 5 min at 37° C., 5% CO₂. The cells were washed with 10 mL DPBS and transferred into a centrifuge tube to spin at 200 g, 3 min. The cell pellets were pooled together in a microcentrifuge tube and the packed cell volume (PCV) was estimated (˜150-200 μL). After resuspending the pellet in 300 μL hypotonic lysis buffer (10 mM HEPES, pH 7.9, 10 mM KCl, 1.5 mM MgCl₂, 1 mM DTT, protease inhibitor (Roche)), the mixture was spun at 100 g for 5 min for initial cell swelling. The supernatant was discarded and the pellet was resuspended in 300 μL hypotonic lysis buffer and placed on ice for 10 min for further swelling. The mixture was transferred to a 2 mL Dounce homogenizer and ground on ice slowly with 45 up-and-down strokes using a type B pestle. Lysis of the cell membrane was checked to be 80-90% complete under the microscope using Trypan Blue as the indicator. The lysate was centrifuged for 5 min at 5,000×g and the supernatant (cytoplasmic fraction) was separated from the crude nuclei pellet (˜100 μL PCV).

The soluble nuclear proteins were isolated following the methods reported by Lyst et al36 with slight modifications. The crude nuclei pellet was first dissolved in 100 μL of low salt extraction buffer (20 mM H EPES, pH 7.6, 10 mM KCl, 1 mM MgCl₂, 0.1% Triton X-100 (vol/vol), protease inhibitors (Roche), 15 mM BME) and dounced with 20 up-and-down strokes using a type B pestle. The lysate was then transferred to a microcentrifuge tube along with an extra 100 μL low salt extraction buffer added to wash the dounce vessel, 250 units of benzonase (Sigma) per 10⁷ nuclei was added to the dounced nuclei for 10 min at RT. High salt extraction buffer (low salt+1 M NaCi) was added dropwise to achieve a final NaCl concentration of 350 mM and the mixture was incubated on a rotating wheel for 45 min at 4° C. The final soluble nuclear supernatant was obtained by centrifuging at 18,000 g for 15 min.

To preserve the interaction of tMeCP2 with the NCoR/SMRT complex36, the nuclear supernatant was buffer exchanged for four times with the coIP buffer (20 mM HEPES, pH 7.6, mN KCl. 150 mM NaCl, 1 mM MgCl₂, 0.1% Triton X-100 (vol/vol), protease inhibitors (Roche), 15 mM BME) using a 3 k MWCO concentrator (Amicon). The final concentration of the nuclear lysate was determined by the Pierce™ 660 nm protein assay, using BSA as standards and diluted to ˜1 mg/mL with the coIP buffer.

### Supplementary Methods 3. LC/MS/MS Analysis

Mass spectrometry was performed by the Proteomics/Mass Spectrometry Laboratory at UC Berkeley. A nano LC column was packed in a 100 μm inner diameter glass capillary with an emitter tip. The column consisted of 10 cm of Polaris c18 5 μm packing material (Varian). The column was loaded by use of a pressure bomb and washed extensively with buffer A (5% acetonitrile/0.02% heptaflurobutyric acid (HBFA)). The column was then directly coupled to an electrospray ionization source mounted on a Thermo-Fisher LTQ XL linear ion trap mass spectrometer. An Agilent 1200 HPLC equipped with a split line so as to deliver a flow rate of 300 nl/min was used for chromatography. Peptides were eluted using a 90 min. gradient from buffer A to 60% Buffer B (80% acetonitrile/0.02% HBFA).

Protein identification and quantification were done with Integrated Proteomics Pipeline (IP2, Integrated Proteomics Applications, Inc. San Diego, CA) using ProLuCID/Sequest, DTASelect2 and Census65-68. Tandem mass spectra were extracted into msl and ms2 files from raw files using RawExtractor69 and were searched against the E. Coli protein database plus sequences of common contaminants with the engineered sequence of the experimental protein added. This database was concatenated to a decoy database in which the sequence for each entry in the original database was reversed70. LTQ data was searched with 3000.0 milli-amu precursor tolerance and the fragment ions were restricted to a 600.0 ppm tolerance. All searches were parallelized and searched on the VJC proteomics cluster. Search space included all fully tryptic peptide candidates with no missed cleavage restrictions. Carbamidomethylation (+57.02146) of cysteine was considered a static modification. We required 1 peptide per protein and both tryptic termini for each peptide identification. The ProLuCID search results were assembled and filtered using the DTASelect program66,67 with a peptide false discovery rate (FDR) of 0.001 for single peptides and a peptide FDR of 0.005 for additional peptides for the same protein. Under such filtering conditions, the estimated false discovery rate was zero for the datasets used.

### REFERENCES

1. Skene, P. J. et al. Neuronal MeCP2 Is Expressed at Near Histone-Octamer Levels and Globally Alters the Chromatin State. Mol. Cell37, 457-468 (2010).
2. Amir, R. E. et al. Rett syndrome is caused by mutations in X-linked MECP2, encoding methyl-CpG-binding protein 2. Nat. Genet.23, 185-188 (1999).
3. Guy, J., Gan, J., Selfridge, J., Cobb, S. & Bird, A. Reversal of Neurological Defects in a Mouse Model of Rett Syndrome. Science315, 1143-7 (2007).
4. Robinson, L. et al. Morphological and functional reversal of phenotypes in a mouse model of Rett syndrome. Brain135, 2699-2710 (2012).
5. Lang, M. et al. Rescue of behavioral and EEG deficits in male and female Mecp2-deficient mice by delayed Mecp2 gene reactivation. Hum. Mol. Genet.23, 303-318 (2014).
6. Tillotson, R. et al. Radically truncated MeCP2 rescues Rett syndrome-like neurological defects. Nature550, 398-401 (2017).
7. Appelbaum, J. S. et al. Arginine topology controls escape of minimally cationic proteins from early endosomes to the cytoplasm. Chem. Biol.19, 819-830 (2012).
8. LaRochelle, J. R., Cobb, G. B., Steinauer, A., Rhoades, E. & Schepartz, A. Fluorescence Correlation Spectroscopy Reveals Highly Efficient Cytosolic Delivery of Certain Penta-Arg Proteins and Stapled Peptides. J. Am. Chem. Soc.137, 2536-2541 (2015).
9. Wissner, R. F., Steinauer, A., Knox, S. L., Thompson, A. D. & Schepartz, A. Fluorescence Correlation Spectroscopy Reveals Efficient Cytosolic Delivery of Protein Cargo by Cell-Permeant Miniature Proteins. ACS Cent. Sci.4, 1379-1393 (2018).
10. Steinauer, A. et al. HOPS-dependent endosomal fusion required for efficient cytosolic delivery of therapeutic peptides and small proteins. Proc. Natl. Acad. Sci.116, 512-521 (2019).
11. Knox, S. L., Wissner, R., Piszkiewicz, S. & Schepartz, A. Cytosolic Delivery of Argininosuccinate Synthetase Using a Cell-Permeant Miniature Protein. ACS Cent. Sci.7, 641-649 (2021).
12. Grimm, N.-B. & Lee, J. T. Selective Xi reactivation and alternative methods to restore MECP2 function in Rett syndrome. Trends Genet. (2022) doi:10.1016/j. tig.2022.01.007.
13. Ehrhart, F. et al. A catalogue of 863 Rett-syndrome-causing MECP2 mutations and lessons learned from data integration. Sci. Data8, 10 (2021).
14. Krishnaraj, R., Ho, G. & Christodoulou, J. RettBASE: Rett syndrome database update. Hum. Mutat. 38, 922-931 (2017).
15. Doudna, J. A. The promise and challenge of therapeutic genome editing. Nature578, 229-236 (2020).
16. Anzalone, A. V., Koblan, L. W. & Liu, D. R. Genome editing with CRISPR-Cas nucleases, base editors, transposases and prime editors. Nat. Biotechnol. 38, 824-844 (2020).
17. Gadalla, K. K. et al. Gene therapy for Rett syndrome: prospects and challenges. Future Neurol. 10, 467-484 (2015).
18. Ramocki, M. B., Tavyev, Y. J. & Peters, S. U. The MECP2 duplication syndrome. Am. J. Med. Genet. A. 152A, 1079-1088 (2010).
19. Sinnett, S. E. & Gray, S. J. Recent Endeavors in MECP2 Gene Transfer for Gene Therapy of Rett Syndrome. Discov. Med. 24, 153-159 (2017).
20. Luoni, M. et al. Whole brain delivery of an instability-prone Mecp2 transgene improves behavioral and molecular pathological defects in mouse models of Rett syndrome. eLife9, e52629 (2020).
21. Sinnett, S. E., Boyle, E., Lyons, C. & Gray, S. J. Engineered microRNA-based regulatory element permits safe high-dose miniMECP2 gene therapy in Rett mice. Brain144, 3005-3019 (2021).
22. Matagne, V. et al. Severe offtarget effects following intravenous delivery of AAV9-MECP2 in a female mouse model of Rett syndrome. Neurobiol. Dis. 149, 105235 (2021).
23. Tillotson, R. & Bird, A. The Molecular Basis of MeCP2 Function in the Brain. J. Mol. Biol. 432, 1602-1623 (2020).
24. Cho, N. H. et al. OpenCell: Endogenous tagging for the cartography of human cellular organization. Science375, eabi6983.
25. Herce, H. D. et al. Cell-permeable nanobodies for targeted immunolabelling and antigen manipulation in living cells. Nat. Chem. 9, 762-771 (2017).
26. Laccone, F. A. Synthetic MeCP2 sequence for protein substitution therapy. (2012).
27. Steinkellner, H. et al. An electrochemiluminescence based assay for quantitative detection of endogenous and exogenously applied MeCP2 protein variants. Sci. Rep. 9, 7929 (2019).
28. Beribisky, A. V. et al. Expression, Purification, Characterization and Cellular Uptake of MeCP2 Variants. Protein J. (2022) doi:10.1007/s10930-022-10054-9.
29. Steinkellner, H. et al. TAT-MeCP2 protein variants rescue disease phenotypes in human and mouse models of Rett syndrome. Int. J. Biol. Macromol. 209, 972-983 (2022).
30. Qian, Z. et al. Discovery and Mechanism of Highly Efficient Cyclic Cell-Penetrating Peptides. Biochemistry55, 2601-2612 (2016).
31. Vivès, E., Brodin, P. & Lebleu, B. A Truncated HIV-1 Tat Protein Basic Domain Rapidly Translocates through the Plasma Membrane and Accumulates in the Cell Nucleus*. J. Biol. Chem. 272, 16010-16017 (1997).
32. Ho, K. L. et al. MeCP2 binding to DNA depends upon hydration at methyl-CpG. Mol. Cell29, 525-531 (2008).
33. Brown, K. et al. The molecular basis of variable phenotypic severity among common missense mutations causing Rett syndrome. Hum. Mol. Genet. 25, 558-570 (2016).
34. Kruusvee, V. et al. Structure of the MeCP2-TBLR1 complex reveals a molecular basis for Rett syndrome and related disorders. Proc. Natl. Acad. Sci. 114, E3243-E3250 (2017).
35. Adams, V. H., McBryant, S. J., Wade, P. A., Woodcock, C. L. & Hansen, J. C. Intrinsic Disorder and Autonomous Domain Function in the Multifunctional Nuclear Protein, MeCP2*. J. Biol. Chem. 282, 15057-15064 (2007).
36. Lyst, M. J. et al. Rett syndrome mutations abolish the interaction of MeCP2 with the NCoR/SMRT co-repressor. Nat. Neurosci. 16, 898-902 (2013).
37. Khrapunov, S. et al. MeCP2 Binding Cooperativity Inhibits DNA Modification-Specific Recognition. Biochemistry55, 4275-4285 (2016).
38. Ghosh, R. P., Horowitz-Scherer, R. A., Nikitina, T., Shlyakhtenko, L. S. & Woodcock, C. L. MeCP2 Binds Cooperatively to Its Substrate and Competes with Histone H1 for Chromatin Binding Sites. Mol. Cell. Biol. 30, 4656-4670 (2010).
39. Khrapunov, S. et al. Unusual Characteristics of the DNA Binding Domain of Epigenetic Regulatory Protein MeCP2 Determine Its Binding Specificity. Biochemistry53, 3379-3391 (2014).
40. Kumar, A. et al. Analysis of protein domains and Rett syndrome mutations indicate that multiple regions influence chromatin-binding dynamics of the chromatin-associated protein MECP2 in vivo. J. Cell Sci. 121, 1128-1137 (2008).
41. Fan, C. et al. Rett mutations attenuate phase separation of MeCP2. Cell Discov. 6, 1-4 (2020).
42. Knox, S. L. et al. Quantification of protein delivery in live cells using fluorescence correlation spectroscopy. Methods Enzymol. 641, 477-505 (2020).
43. Schwille, P. & Haustein, E. Fluorescence Correlation Spectroscopy. 33.
44. Zhao, Z. W. et al. Quantifying transcription factor-DNA binding in single cells in vivo with photoactivatable fluorescence correlation spectroscopy. Nat. Protoc. 12, 1458-1471 (2017).
45. Okada, S. et al. Flow cytometric sorting of neuronal and glial nuclei from central nervous system tissue. J. Cell. Physiol. 226, 552-558 (2011).
46. van der Beek, J., Jonker, C., van der Welle, R., Liv, N. & Klumperman, J. CORVET, CHEVI and HOPS—multisubunit tethers of the endo-lysosomal system in health and disease. J. Cell Sci. 132, jcs189134 (2019).
47. Shvarev, D. et al. Structure of the lysosomal membrane fusion machinery. 2022.05.05.490745 (2022) doi:10.1101/2022.05.05.490745.
48. Perini, E. D., Schaefer, R., Stöter, M., Kalaidzidis, Y. & Zerial, M. Mammalian CORVET Is Required for Fusion and Conversion of Distinct Early Endosome Subpopulations. Traffic 15, 1366-1389 (2014).
49. Trofimenko, E. et al. Genetic, cellular, and structural characterization of the membrane potential-dependent cell-penetrating peptide translocation pore. eLife10, e69832 (2021).
50. Kudo, S. et al. Heterogeneity in residual function of MeCP2 carrying missense mutations in the methyl CpG binding domain. J. Med. Genet. 40, 487-493 (2003).
51. Kudo, S. et al. Functional analyses of MeCP2 mutations associated with Rett syndrome using transient expression systems. Brain Dev. 23, S165-S173 (2001).
52. Kitsera, N., Khobta, A. & Epe, B. Destabilized green fluorescent protein detects rapid removal of transcription blocks after genotoxic exposure. BioTechniques 43, 222-227 (2007).
53. Kaur, G. et al. Probing transcription factor diffusion dynamics in the living mammalian embryo with photoactivatable fluorescence correlation spectroscopy. Nat. Commun. 4, 1637 (2013).
54. Ziegler, A. & Seelig, J. High Affinity of the Cell-Penetrating Peptide HIV-1 Tat-PTD for DNA. Biochemistry46, 8138-8145 (2007).
55. Lamonica, J. M. et al. Elevating expression of MeCP2 T158M rescues DNA binding and Rett syndrome-like phenotypes. J. Clin. Invest. 127, 1889-1904.
56. Brock, D. J., Kondow-McConaghy, H. M., Hager, E. C. & Pellois, J.-P. Endosomal Escape and Cytosolic Penetration of Macromolecules Mediated by Synthetic Delivery Agents. Bioconjug. Chem. 30, 293-304 (2019).
57. Deprey, K., Becker, L., Kritzer, J. & Plückthun, A. Trapped!A Critical Evaluation of Methods for Measuring Total Cellular Uptake versus Cytosolic Localization. Bioconjug. Chem. 30, 1006-1027 (2019).
58. Dou, D., Revol, R., Östbye, H., Wang, H. & Daniels, R. Influenza A Virus Cell Entry, Replication, Virion Assembly and Movement. Front. Immunol. 9, (2018).
59. Hou, X., Zaks, T., Langer, R. & Dong, Y. Lipid nanoparticles for mRNA delivery. Nat. Rev. Mater. 6, 1078-1094 (2021).
60. Greenfield, N. J. Using circular dichroism spectra to estimate protein secondary structure. Nat. Protoc. 1, 2876-2890 (2006).
61. Ghosh, R. P. et al. Unique Physical Properties and Interactions of the Domains of Methylated DNA Binding Protein 2. Biochemistry49, 4395-4410 (2010).
62. Ghosh, R. P., Horowitz-Scherer, R. A., Nikitina, T., Gierasch, L. M. & Woodcock, C. L. Rett Syndrome-causing Mutations in Human MeCP2 Result in Diverse Structural Changes That Impact Folding and DNA Interactions. J. Biol. Chem. 283, 20523 (2008).
63. Khrapunov, S. et al. MeCP2 Binding Cooperativity Inhibits DNA Modification-Specific Recognition. Biochemistry55, 4275-4285 (2016).
64. Eccles, L. J., Lomax, M. E. & O'Neill, P. Hierarchy of lesion processing governs the repair, double-strand break formation and mutability of three-lesion clustered DNA damage. Nucleic Acids Res. 38, 1123-1134 (2010).
65. Xu, T. et al. ProLuCID: an improved SEQUEST-like algorithm with enhanced sensitivity and specificity. J. Proteomics 129, 16-24 (2015).
66. Cociorva, D., L Tabb, D. & Yates, J. R. Validation of tandem mass spectrometry database search results using DTASelect. Curr. Protoc. Bioinforma. Chapter 13, Unit 13.4 (2007).
67. Tabb, D. L., McDonald, W. H. & Yates, J. R. DTASelect and Contrast: tools for assembling and comparing protein identifications from shotgun proteomics. J. Proteome Res. 1, 21-26 (2002).
68. Park, S. K., Venable, J. D., Xu, T. & Yates, J. R. A quantitative analysis software tool for mass spectrometry-based proteomics. Nat. Methods 5, 319-322 (2008).
69. McDonald, W. H. et al. MS1, MS2, and SQT-three unified, compact, and easily parsed file formats for the storage of shotgun proteomic spectra and identifications. Rapid Commun. Mass Spectrom. RCM18, 2162-2168 (2004).
70. Peng, J., Elias, J. E., Thoreen, C. C., Licklider, L. J. & Gygi, S. P. Evaluation of Multidimensional Chromatography Coupled with Tandem Mass Spectrometry (LC/LC-MS/MS) for Large-Scale Protein Analysis: The Yeast Proteome. J. Proteome Res. 2, 43-50 (2003).
71. Michelman-Ribeiro, A. et al. Direct Measurement of Association and Dissociation Rates of DNA Binding in Live Cells by Fluorescence Correlation Spectroscopy. Biophys. J. 97, 337-346 (2009).

TABLE 1
Relevant primers and sequences
Cloning to generate tMeCP2 variants: DNA sequence

| | |
|---|---|
| ZF5.3- | TGAGCGGATAACAATTCCCCTCTAGAAATAATTTT |
| tMeCP2(ANC)- | GTTTAACTTTAAGAAGGAGATATACCATGTACAGC |
| LPETGG-Strep | TGCAACGTGTGTGGGAAAGCTTTTGTACTTTCGCG |
| protein | CCACTTGAACCGCCACTTACGTGTACATCGTCGCG |
| | CGACTGGTAGCGGGATGGTAGCTGGCATGCTGGGT |
| | CTTCGCGAAGAGAAACCAGCCGTTCCGGAAGCATC |
| | GGCGTCGCCCAAGCAACGTCGCAGCATCATCCGCG |
| | ACCGCGGCCCCATGTACGATGATCCGACTTTACCT |
| | GAAGGTTGGACACGTAAACTGAAGCAACGTAAGAG |
| | CGGGCGTTCAGCGGGGAAGTATGATGTTTATTTGA |
| | TTAACCCACAGGGGAAGGCTTTCCGCTCTAAGGTT |
| | GAGCTTATCGCCTACTTCGAGAAAGTGGGAGATAC |
| | CTCGCTGGATCCGAACGATTTCGATTTTACAGTCA |
| | CAGGCCGTGGCTCCCCATCCCGCCGCGAGCAGAAG |
| | CCTCCGAAAAAACCAAAATCTCCTAAGGCGCCAGG |
| | CACTGGACGTGGTCGCGGGCGTCCTAAGGGCAGCG |
| | GTACCGGACGCCCAAAAGCAGCTGCCTCCGAAGGA |
| | GTACAAGTGAAACGTGTTTTAGAAAAGAGTCCAGG |
| | AAAACTTGTTGTGAAAATGCCCTTTCAGGCGAGCC |
| | CGGGGGGCAAAGGCGAGGGCGGCGGCGCCACAACA |
| | TCTGCTCAGGTAATGGTTATCAAACGTCCTGGCCG |
| | TAAGCGTAAAGCCGAAGCGGACCCTCAAGCAATTC |
| | CAAAGAAACGCGGTCGTAAACCAGGCTCGGTAGTA |
| | GCCGCTGCGGCGGCTGAAGCTAAAAAGAAGGCCGT |
| | TAAGGAGTCTAGCATTCGCAGTGTGCACGAGACAG |
| | TTTTACCGATCAAGAAACGTAAGACTCGTGAGACC |
| | GTTGGCTCTAGCGGGTCCTCAGGTCTTCCGGAAAC |
| | TGGAGGGTGGAGTCACCCACAATTCGAGAAATAAC |
| | GGATCCGAATTCGAGCGCCGTCGACAAGCT |
| | (SEQ ID NO: 1) |
| Generating | TGAGCGGATAACAATTCCCCTCTAGAAATAATTTT |
| tMeCP2(ANC)- | GTTTAACTTTAAGAAGGAGATATACCATGGTAGCT |
| LPETGG-Strep | GGCATGCTGGGTCTTCGCGAAGAGAAACCAGCCGT |
| from ZF5.3- | TCCGGAAGCATCGGCGTCGCCCAAGCAACGTCGCA |
| tMeCP2(ANC)- | GCATCATCCGCGACCGCGGCCCCATGTACGATGAT |
| LPETGG-Strep | CCGA |
| | (SEQ ID NO: 2) |
| Generating Tat- | TGAGCGGATAACAATTCCCCTCTAGAAATAATTTT |
| tMeCP2(ANC)- | GTTTAACTTTAAGAAGGAGATATACCATGTACGGT |
| LPETGG-Strep | CGCAAAAAACGCCGCCAACGTCGTCGCGGTTCCGG |
| from ZF5.3- | TATGGTAGCTGGCATGCTGGGTCTTCGCGAAGAGA |
| tMeCP2(ANC)- | AACCAGCCGTTCCGGAAGCATCGGCGTCGCCCAAG |
| LPETGG-Strep | CAACGTCGCAGCATCATCCGCGACCGCGGCCCCAT |
| | GTACGATGATCCGA |
| | (SEQ ID NO: 3) |
| Primers for Q5 | F: TTTCGATTTTatgGTCACAGGCCGTGGC |
| Site-Directed | (SEQ ID NO: 4) |
| Mutagenesis to | R: TCGTTCGGATCCAGCGAG |
| generate ZF5.3- | (SEQ ID NO: 5) |
| tMeCP2(ANC)- | |
| T158M-LPETGG- | |
| Strep | |
| Primers for Q5 | F: GACAGTTTTActgATCAAGAAACGTAAG |
| Site-Directed | (SEQ ID NO: 6) |
| Mutagenesis to | R: TCGTGCACACTGCGAATG |
| generate ZF5.3- | (SEQ ID NO: 7) |
| tMeCP2(ANC)- | |
| P302L-LPETGG- | |
| Strep | |

Protein sequences of tMeCP2 variants

| | |
|---|---|
| tMeCP2(ANC)- | MVAGMLGLREEKPAVPEASASPKQRRSIIRDRGPM |
| LPETGG-Strep | YDDPTLPEGWTRKLKQRKSGRSAGKYDVYLINPQG |
| | KAFRSKVELIAYFEKVGDTSLDPNDFDFTVTGRGS |
| | PSRREQKPPKKPKSPKAPGTGRGRGRPKGSGTGRP |
| | KAAASEGVQVKRVLEKSPGKLVVKMPFQASPGGKG |
| | EGGGATTSAQVMVIKRPGRKRKAEADPQAIPKKRG |
| | RKPGSVVAAAAAEAKKKAVKESSIRSVHETVLPIK |
| | KRKTRETVGSSGSSGLPETGGWSHPQFEK* |
| | (SEQ ID NO: 8) |
| ZF5.3- | MYSCNVCGKAFVLSRHLNRHLRVHRRATGSGMVAG |
| tMeCP2(ANC)- | MLGLREEKPAVPEASASPKQRRSIIRDRGPMYDDP |
| LPETGG-Strep | TLPEGWTRKLKQRKSGRSAGKYDVYLINPQGKAFR |
| | SKVELIAYFEKVGDTSLDPNDFDFTVTGRGSPSRR |
| | EQKPPKKPKSPKAPGTGRGRGRPKGSGTGRPKAAA |
| | SEGVQVKRVLEKSPGKLVVKMPFQASPGGKGEGGG |
| | ATTSAQVMVIKRPGRKRKAEADPQAIPKKRGRKPG |
| | SVVAAAAAEAKKKAVKESSIRSVHETVLPIKKRKT |
| | RETVGSSGSSGLPETGGWSHPQFEK* |
| | (SEQ ID NO: 9) |
| Tat-tMeCP2(ANC)- | MYGRKKRRQRRRGSGMVAGMLGLREEKPAVPEASA |
| LPETGG-Strep | SPKQRRSIIRDRGPMYDDPTLPEGWTRKLKQRKSG |
| | RSAGKYDVYLINPQGKAFRSKVELIAYFEKVGDT |
| | SLDPNDFDFTVTGRGSPSRREQKPPKKPKSPKAPG |
| | TGRGRGRPKGSGTGRPKAAASEGVQVKRVLEKSPG |
| | KLVVKMPFQASPGGKGEGGGATTSAQVMVIKRPGR |
| | KRKAEADPQAIPKKRGRKPGSVVAAAAAEAKKKAV |
| | KESSIRSVHETVLPIKKRKTRETVGSSGSSGLPET |
| | GGWSHPQFEK* |
| | (SEQ ID NO: 10) |
| ZF5.3- | MYSCNVCGKAFVLSRHLNRHLRVHRRATGSGMVAG |
| tMeCP2(ANC)- | MLGLREEKPAVPEASASPKQRRSIIRDRGPMYDDP |
| T158M-LPETGG- | TLPEGWTRKLKQRKSGRSAGKYDVYLINPQGKAFR |
| Strep | SKVELIAYFEKVGDTSLDPNDFDFMVTGRGSPSRR |
| | EQKPPKKPKSPKAPGTGRGRGRPKGSGTGRPKAAA |
| | SEGVQVKRVLEKSPGKLVVKMPFQASPGGKGEGGG |
| | ATTSAQVMVIKRPGRKRKAEADPQAIPKKRGRKPG |
| | SVVAAAAAEAKKKAVKESSIRSVHETVLPIKKRKT |
| | RETVGSSGSSGLPETGGWSHPQFEK* |
| | (SEQ ID NO: 11) |
| ZF5.3- | MYSCNVCGKAFVLSRHLNRHLRVHRRATGSGMVAG |
| tMeCP2(ANC)- | MLGLREEKPAVPEASASPKQRRSIIRDRGPMYDDP |
| P302L-LPETGG- | TLPEGWTRKLKQRKSGRSAGKYDVYLINPQGKAFR |
| Strep | SKVELIAYFEKVGDTSLDPNDFDFTVTGRGSPSRR |
| | EQKPPKKPKSPKAPGTGRGRGRPKGSGTGRPKAAA |
| | SEGVQVKRVLEKSPGKLVVKMPFQASPGGKGEGGG |
| | ATTSAQVMVIKRPGRKRKAEADPQAIPKKRGRKPG |
| | SVVAAAAAEAKKKAVKESSIRSVHETVLLIKKRKT |
| | RETVGSSGSSGLPETGGWSHPQFEK* |
| | (SEQ ID NO: 12) |

DNA probes used for fluorescence polarization

| | |
|---|---|
| Methylated | Strand 1: |
| | 5′/56-FAM/CCCTATAA/Me-dC/GGAATTCATAATG-3′ |
| | (SEQ ID NO: 13) |
| | Strand 2: |
| | 5′-CATTATGAATTC/Me-dC/GTTATAGGG-3′ |
| | (SEQ ID NO: 14) |
| Non-methylated | Strand 1: |
| | 5′/56-FAM/CCCTATAACGGAATTCATAATG-3′ |
| | (SEQ ID NO: 15) |
| | Strand 2: |
| | 5′-CATTATGAATTCCGTTATAGGG-3′ |
| | (SEQ ID NO: 16) |

siRNA used in RNAi

| | |
|---|---|
| RISC-free | siGENOME RISC-Free Control siRNA: |
| knockdown | D-001220-01 (Dharmacon) |
| KIF11 knockdown | siGENOME human SMARTpool siRNA KIF11: |
| | D-001220-01 (Dharmacon) |
| VPS39 knockdown | siGENOME human SMARTpool siRNA VPS39: |
| | M-014052-01 (Dharmacon) |
| TGFBRAP1 | siGENOME human SMARTpool siRNA TGFBRAPI: |
| knockdown | M-006903-01 (Dharmacon) |

Gene-specific primers for RT-qPCR

| | |
|---|---|
| GAPDH | Prime Time qPCR primers Hs.PT. |
| amplification | 39a.22214836 (IDT) |
| TGFBRAP1 | Forward: |
| | CTGACCACTCAGTACATCATCC (SEQ ID NO: 17) |
| amplification | Reverse: |
| | CTCCTGTCTCCCTATCCTCTT (SEQ ID NO: 18) |
| VPS39 | Forward: |
| | AGGGTCTGGCTATTCCTTATCT (SEQ ID NO: 19) |
| amplification | Reverse: |
| | CCTTGCACCTTCTCACAGTATAG (SEQ ID NO: 20) |

Cloning to generate reporter plasmids for
in cellulo transcription assay

| | |
|---|---|
| Primers to amplify | Forward: |
| mNeonGreen gene | AGCGAGGAAGGTCAGAAGCTTGGCAATCCGGTACT |
| | GGCCACCATGGTGAGCAAGGGCGA |
| | (SEQ ID NO: 21) |
| | Reverse: |
| | GGCGGAAAGCCGTGAGAATTCGCCTTGTACAGCTC |
| | GTCCATGC (SEQ ID NO: 22) |
| Primers to amplify | Forward: |
| SNRPN promoter | CCGGTACCTGAGCTCGCTAGCTTTCTGTACAGAAT |
| | TTTAAGTATCTGACAATTT (SEQ ID NO: 23) |
| | Reverse: |
| | CAGTACCGGATTGCCAAGCTTCTGACCTTCCTCGC |
| | TCCATTG (SEQ ID NO: 24) |

1. A cell-penetrant ZF5.3-MeCP2 fusion protein comprising a ZF5.3 moiety and a methyl-CpG binding protein 2 (MeCP2) moiety, the MeCP2 moiety comprising a methyl-CpG binding domain (MBD) and a NCoR/SMRT interaction domain (NID).

2. The cell-penetrant ZF5.3-MeCP2 fusion protein of 1, wherein the ZF5.3 moiety is N-terminal with respect to the MeCP2 moiety.

3. The cell-penetrant ZF5.3-MeCP2 fusion protein of claim 1, wherein the MBD and NID comprise residues 72-173 and 272-312 of MeCP2, respectively, with respect to the mouse e2 isoform.

4. The cell-penetrant ZF5.3-MeCP2 fusion protein of claim 1, wherein the ZF5.3 moiety is N-terminal with respect to the MeCP2 moiety, and the MBD and NID comprise residues 72-173 and 272-312 of MeCP2, respectively, with respect to the mouse e2 isoform.

5. The cell-penetrant ZF5.3-MeCP2 fusion protein of claim 1, wherein the fusion protein reaches the nucleus of the cell intact and transcriptionally active, as measured in human osteosarcoma (clone Saos-2) or hamster chinese ovary (clone CHO-K1) cells.

6. The cell-penetrant ZF5.3-MeCP2 fusion protein of claim 4, wherein the fusion protein reaches the nucleus of the cell intact and transcriptionally active, as measured in human osteosarcoma (clone Saos-2) or hamster chinese ovary (clone CHO-K1) cells.

7. The cell-penetrant ZF5.3-MeCP2 fusion protein of claim 5, wherein the fusion protein reaches the nucleus of the cell at at least 100 nM.

8. The cell-penetrant ZF5.3-MeCP2 fusion protein of claim 6, wherein the fusion protein reaches the nucleus of the cell at at least 100 nM.

9. The cell-penetrant ZF5.3-MeCP2 fusion protein of claim 5, wherein the fusion protein reaches the nucleus of the cell at at least 500 nM.

10. The cell-penetrant ZF5.3-MeCP2 fusion protein of claim 6, wherein the fusion protein reaches the nucleus of the cell at at least 500 nM.

11. A method of introducing MeCP2 activity in a host in need thereof, comprising delivering the MeCP2 activity in the form of the cell-penetrant ZF5.3-MeCP2 fusion protein of claim 1.

12. A method of treating Rett syndrome (RTT), comprising administering to a person in need thereof the cell-penetrant ZF5.3-MeCP2 fusion protein of claim 1.
